(19) United States
(12) Patent Application Publication
(10) Pub. No.: US 2011/0020950 A1
(43) Pub. Date: Jan. 27, 2011

(54) SCAFFOLD FOR COMPOSITE BIOMIMETIC MEMBRANE

(75) Inventors: Jörg Vogel, Copenhagen (DK); Mark Edward Perry, Holte (DK); Claus Hélix Nielsen, Hoje-Taastrup (DK); Jesper Søndergaard Hansen, Soborg (DK); Peter Holme Jensen, Copenhagen (DK); Oliver Geschke, Kgs. Lyngby (DK); Pierre-Yves Bolinger, Dubendorf (CH)

Correspondence Address: CLARK & ELBING LLP, 101 FEDERAL STREET, BOSTON, MA, 02110, US

(73) Assignee: AQUAPORIN A/S

(21) Appl. No.: 12/747,074

(22) PCT Filed: Dec. 11, 2008
(86) PCT No.: PCT/DK08/50303

§ 371 Date: Oct. 12, 2010

Related U.S. Application Data
(60) Provisional application No. 61/082,087, filed on Jul. 18, 2008.

(30) Foreign Application Priority Data
Dec. 11, 2007 (KR) PA 2007 01771

(51) Int. Cl. G01N 33/566; B01J 19/00; C12Q 1/48; B29C 35/08

(52) U.S. Cl. 436/501; 422/310; 422/68.1; 435/15; 264/400

(57) ABSTRACT

Disclosed herein is a membrane scaffold comprising a planar material having a hydrophobic surface and a functional area comprising a plurality of apertures. The apertures have a diameter of from about 80 μm to about 3000 μm and the rims of the apertures comprise bulges extending above and/or below the surface level of the planar material. The membrane scaffold is useful in the preparation of a composite biomimetic membrane wherein functional channel forming molecules have been incorporated in said membrane.

### FIELD OF THE INVENTION

The present invention relates to a planar hydrophobic membrane scaffold having multiple apertures suitable for the formation of biomimetic membranes, a method for producing the membrane scaffold, a composite biomimetic membrane comprising said scaffold, a filtration device comprising the composite biomimetic membrane, as well as a method of preparing said composite biomimetic membrane.

### BACKGROUND

Membranes comprising an artificial lipid bilayer with incorporated functional molecules, such as ion channel peptides and transmembrane proteins are useful in a diverse range of technical applications. A common theme for such membranes is the need for stability of the membranes over time and against mechanical, electrical and chemical impacts. Planar lipid bilayers are usually supported in apertures or perforations of a scaffold or septum separating two solution compartments. Various hydrophobic materials have been used as scaffolds, including an amorphous Teflon® (Teflon® AF) film, cf. Mayer et al. (Bio physical Journal Vol 85, October 2003, 2684-2695). Various methods of fabrication of such a scaffold having a single aperture or a plurality of apertures have been described, e.g. puncturing the scaffold film with a needle, or a heated wire and various other mechanical methods. It is reported that useful materials for the preparation of lipid or amphiphilic bilayer membranes are Teflon films and other membrane materials with hydrophobic surface properties. Current methods of preparing BLMs include the solvent free Folded bilayers method described by Montal & Muller (1972, PNAS, 69:3561-3566) which require small apertures (<100 μM) and the solvent containing Painted bilayers method described by Muller & Rodin (1969, Cur. Top. Bioeng. 3:157-249) which is optimal for apertures of up to 400 μm. Both methods are useful in the preparation of a BLM in a single aperture or a small number of apertures such as less than 5 in a hydrophobic partition, but they are not straight forward to scale into multi aperture partitions. Establishing a folded membrane often requires multiple lowerings and raisings of the aqueous solutions which may compromise the simultaneous formation of a plurality of membrane units. Formation of painted membranes requires manual prepainting of the single aperture, which, when scaled up will lead to considerable variation in painting quality.

Since the discovery of the aquaporin water transport proteins distinguished by their ability to selectively transport H₂O molecules across biological membranes there has been a certain interest in devising an artificial water membrane incorporating these proteins, cf. published US Patent Application No. 2004/0049230 “Biomimetic membranes” which aims to describe how water transport proteins are embedded in a membrane to enable water purification. The preferred form described has the form of a conventional filter disk. To fabricate such a disk, a 5 nm thick monolayer of synthetic triblock copolymer and aquaporin protein is deposited on the surface of a 25 mm commercial ultrafiltration disk using Langmuir-Blodgett transfer. The monolayer on the disk is then crosslinked using UV light to the polymer to increase its durability. It has been suggested that a water purification technology could be created by expressing the aquaporin protein into lipid bilayer vesicles and cast these membranes on porous supports, cf. James R. Swartz, home page at http://cheme.stanford.edu/faculty/jswartz.html

WO 2006/122566 discloses a membrane for filtering of water comprising a sandwich construction having at least two permeable support layers separated by at least one lipid bilayer comprising functional aquaporin water channels. WO 2006/122566 also discloses a hydrophobic film comprising evenly distributed perforations having a uniform shape and size, where the lipid bilayer is formed in the perforations. It is stated that the hydrophobic material has a degree of hydrophobicity corresponding to a contact angle of at least 100° between a droplet of de-ionised water and the hydrophobic material, where the contact angle measurement is performed at 20° C. and atmospheric pressure, but higher degrees of hydrophobicity are preferred, such as those corresponding to contact angles of at least 105°, 110°, or 120°. A preferred hydrophobic material is Teflon. The polymer film comprises multiple perforations, wherein said perforations are evenly distributed in the film and substantially all of the same geometric shape in the intermediate plane between the 2 surfaces of the film. The perforations typically have a maximum cross-sectional length in the nm to mm range, such as in the μm range, and the films as such typically have a thickness in the μm to mm range. The geometric shape of the perforations is selected from circular and elliptical, and it is stated that both shapes are easily obtainable when using laser equipment for introducing the perforations in the film. For instance, circular apertures can be obtained by using a stand-still laser beam, whereas movement of the film relative to the laser beam (either by moving the film or the laser beam) during exposure would provide an elliptical perforation. The hydrophobic polymer films of this prior art contains multiple perforations or apertures which are suitable for the support of a biomimetic membrane, such as a bilayer lipid membrane. While it is preferred that the apertures' geometric shape is circular corresponding to a cylindrical form or ellipsoidal corresponding to an elliptic cylinder (rod-like shape) there is a lack of specific teaching as to a preferred or optimal shape of the aperture rim. The present inventors have realised that the characteristics of the aperture rim is highly correlated with the longevity of the biomimetic membranes formed in said apertures and besides, that use of the ETFE material for the formation of aperture arrays enables preparation of highly stable composite biomimetic membranes.

### SUMMARY OF THE INVENTION

The present invention relates to a membrane scaffold comprising a planar material having a hydrophobic surface and a functional area comprising a plurality of apertures, wherein the apertures have a diameter of from about 80 μm to about 3000 μm, preferably 800 μm and the rims of the apertures comprise bulges extending above and/or below the surface level of the planar material. The bulging of the rims may contribute to the stabilisation of the scaffold material and/or bilayer membranes, such as BLMs, subsequently formed in the scaffold. Thus, due to the bulging rims it is possible to position the plurality of apertures close to each other without risking the breakage of the membrane scaffold. Thereby, the present invention offers the advantage of obtaining a highly effective membrane area, i.e. a high perforation area in the functional area, without destabilisation of the membrane scaffold during operation. In addition, the functional scaffold area can be up-scaled to 20 cm² or more even when fabricated in very thin planar material of less than 200 μm thickness.

The hydrophobic surface of the membrane scaffold usually has a water contact angle larger than 90°, such as larger than about 100°. Specific examples of the planar material include a fluoropolymer film, such as a Teflon (polytetrafluoroethylen, PTFE) or a polyethylenetetrafluoroethylene (ETFE) film including suitable derivatives thereof.

The functional area comprises a plurality of apertures and may be formed using an optically induced or stimulated thermal process. The cross section of said apertures in said planar material is essentially of a circular or approximately circular shape viewed from above and has an essentially perpendicular axis relative to the plane of said planar material. The apertures are characterized by rims, which are smooth and expand to bulges, which are formed onto the surface of said planar material. The functional area of the membrane scaffold may be optimized to obtain a perforation as high as possible while maintaining the physical integrity during operation of the ensuing membrane.

In a certain aspect of the invention, the perforated area of the functional area is 20% or above. In preferred embodiments the perforated area covers from about 30% to about 60% of said functional area. In the membrane scaffold according to the invention the aperture rim may have a toroidal bulging which contributes to stabilization of the membranes formed in the apertures. It is presently believed that the bulging rims of the apertures are able to support a sufficiently large torus (or annulus) of fluid amphiphilic lipid membrane forming solution, which probably participates in stabilizing the bilayer membrane.

The diameter of the apertures may vary according to the design needs within the range of 80 to 800 μm and they may be produced with a diameter of up to 3000 μm. Experiments have shown that bilayer lipid membranes form easily in apertures of 200 μm to about 300 μm, especially 250 μm to about 450 μm. Typically the membranes last from 24 hours to 13 days. The number of apertures in the functional area is normally 25 or more to obtain a high effective membrane area. In a preferred aspect of the invention, the number of apertures is 64 or above, such as 100 or above. The apertures are usually distributed in a certain pattern in the functional area, such as a hexagonal pattern, a triangular pattern or a rectangular or square pattern. A regular pattern may be preferred in the scaffolds of the invention due to the ease of manufacturing and reproducibility.

The bulges of the rims extend above the surface of the planar material to obtain a higher physical stability. When measuring the bulge heights using atomic force microscopy they are found to extend 6 μm or more above the level of the planar material. A typical range of bulge heights is from about 6 to about 20 μm. In a preferred aspect of the invention, the bulges of neighbouring apertures may be merged into a common bulge. In this instance, which is found between apertures in the inner rows and columns of the scaffold array, the bulges can generally be higher, e.g. measured up to about 15 μm (Height of merged bulges (triple point)=15.3±4.4 μm, and height of merged bulges (center)=12.7±6.4 μm) for specific 3×3 aperture scaffolds produced in Tefzel 200LZ having a center-to-center distance of 400 μm and an aperture diameter of 295-300 μm, and where the outer, non-merged bulges were measured to be 9.7±1.7 μm. The merging of the bulges may be entirely to obtain a single bulge (center) between neighbouring bulges or partly according to which the individual bulges may still be discerned (triple point). The aperture rims are usually smooth to support the longevity of the biomimetic membrane formed in the apertures. The center-to-center distance of neighbouring apertures may vary in a functional area. To obtain a high aperture density, the distance is usually not below 120 μm nor above 4000 μm. In a preferred aspect, the center-to-center distance is from about 150 μm to about 500 μm. The planar material may have any suitable thickness.

Generally, it is suitable to use a planar material that has a thickness of from about 25 μm to about 200 μm. A preferred ETFE film has a thickness of about 50 μm to about 75 μm. The hydrophobic surface of the membrane scaffold material may be covered with a coating, e.g. deposited through chemical vapour deposition. The coating may serve various functions, such as enhancement of the formation of the membrane, stabilisation of the membrane, improvement of the smoothness of the surface, and reinforcement of the membrane scaffold. The coating may be applied onto the scaffold membrane and adhered thereto or chemically bonded to the surface of the scaffold membrane. The coating may for instance be a homogeneous layer of a hydrophobic substance when a lipid bilayer membrane is intended. The initial pre-treatment with a lipid solution ensures a higher stability of the membrane. The lipid layer may be applied by any suitable means including spraying and painting. Usually, the lipid solution is applied several times to the scaffold with intermediate drying periods. According to another embodiment, a compound is chemically bonded to the surface, e.g. by a covalent bonding. As an example, the hydrophobic surface of the planar material may be modified by reaction with sodium naphthalenide as disclosed by Ayurova, O. Zh., et al., Russian Journal of Applied Chemistry, vol. 78, No. 5, 2005, pp. 850-852.

In addition, the present invention relates to a method for producing the membrane scaffold. The method includes the steps of:
a. providing a planar material having a hydrophobic surface,
b. subjecting a spot of a functional area of the planar material to a laser beam having a wave length absorbed by the planar material for a time and a power sufficient for the planar material to melt and/or vaporize at said spot,
c. allowing the melted material to solidify around the spot, thereby forming a bulging aperture rim,
d. displacing the planar material or the laser beam to another spot of the functional area and
e. repeating steps b. to d. until a plurality of apertures have been formed.

The method for production of the scaffold includes the use of a laser beam, which preferably is provided by a CO₂ laser, and the planar material is preferably a polyethylenetetrafluoroethylene (ETFE) film or a derivative of ETFE.

In this method it is further preferred that a neighbouring spot is subjected to a laser beam before solidification of the melted material of a previous spot and/or wherein the apertures initially produced are receiving a higher spot lase duration and/or a higher intensity than the subsequently produced apertures. The laser beam and/or the planar material in step d is preferably displaced about 150 μm to about 500 μm.

The planar material partly melts when impacted by the laser beam. The melted material subsequently solidifies, preferably to form smooth bulges. To obtain a merging of bulges it may be preferred that a neighbouring spot is subjected to a laser beam before solidification of the melted material of a pervious spot. The laser beam may have any suitable power (or intensity) and spot lase duration for the apertures of the invention to be obtained. In a preferred embodiment the laser beam is operated at a power of about 3 W to about 8.5 W, and the laser beam is preferably operated at a spot lase duration of between 1 and 7 ms. In accordance with the desired specifications of the bulges the off vector delay is of 1 μs to 1000 μs. The spot lase duration and/or the power may be varied during the production of the functional area. Thus, according to a preferred aspect, the apertures initially produced are receiving a higher spot lase duration and/or a higher power than the apertures subsequently produced to obtain a uniform appearance of the scaffold.

The membrane scaffold is especially useful in the preparation of a composite biomimetic membrane where an amphiphilic membrane forming composition has been deposited in said apertures to form the membrane wherein functional molecules, such as channel forming molecules, e.g. certain peptides or peptide like molecules including amphotericin B, alamethicin, valinomycin, gramicidin A and their dimers, oligomers and analogues thereof; or transmembrane proteins, e.g. aquaporin water channels, Fas protein, DsbB, CFTR, alpha-haemolysin, VDAC, and OmpG, are incorporated.

Thus, the present invention also relate to a composite biomimetic membrane comprising the membrane scaffold described above, and a biomimetic membrane provided in the apertures, wherein functional channel forming molecules have been incorporated in the membrane. In a preferred aspect of the invention, the channel-forming molecule is selected among the aquaporin water channels to make it possible to obtain a composite biomimetic membrane useful in a filtration device for purification of a water source or a liquid, aqueous medium. Other useful applications include a biosensor or for high throughput screening of ligands. The present inventors have found that the membrane scaffold described herein is especially suitable for the formation of bilayer lipid membranes in its apertures, and that said membranes have an increased longevity compared to membranes of the prior art. The biomimetic membrane of the invention is suitable for incorporation of biomolecules that are naturally membrane-bound, e.g. aquaporins, or for incorporation of artificial molecules. The composite biomimetic membranes comprising aquaporins are suitable for transporting water from one side of the membrane to the other side, e.g. when driven by a pressure gradient. The ability to transport water may be utilized in a filtration device for preparing essentially pure water. Other embodiments of the composite biomimetic membrane are suitable as biosensors or for high troughput screening of transmembrane protein ligands. The channel-forming molecules cover in a preferred aspect at least 1% of the membrane surface. Suitably, the membrane is covered with 1 to 10% of the channel-forming molecules.

The invention relates in a further aspect to a filtration device for filtering essentially pure water comprising a composite biomimetic membrane comprising aquaporin water channels as described above. The advantages of using the composite membrane in said filtering device or other applications where upscale is an advantage is closely related to the possibility of up-scaling the functional membrane area by the manufacturing of large, flexible, and relatively thin sheets having a large multitude of discrete membrane units. In addition, the composite membrane ensures that filtering ability is maintained even though one or more discrete membrane units have failed. This situation may especially apply to a filtration device having multi layer stacking of the individual composite membranes or 2D-aperture-arrays.

Furthermore, the invention relates to a novel method of forming auto-painted membranes (APM) in said scaffold to prepare a composite biomimetic membrane, and a chamber for the preparation and holding of said composite biomimetic membrane. Surprisingly, the inventors have found that the principle of the APM technique which uses a narrow reservoir of a concentrated, limited volume of amphiphilic membrane forming solution (e.g. DPhPC lipid mixed with an apolar solvent, e.g. a hydrocarbon solvent) in direct connection with a buffer volume on the front side (cis chamber) of the vertically positioned scaffold/partition is able to facilitate preparation of a composite biomimetic membrane. When raising said buffer solution the amphiphilic membrane forming solution will be raised completely past the scaffold (Teflon partition) and in the process be deposited into the multiple apertures, which have been prepainted with a solution of amphiphilic substance in an apolar solvent, to create a composite membrane in said scaffold apertures. The hydrophobic nature of the scaffold surface ensures deposition of the apolar membrane forming solution into said multiple apertures. An optional feature of the APM method is that the composite membrane is supported and stabilized on the back side (trans chamber) by a preferably hydrophilic, porous support material that allows fluid connection between the membrane and the buffer solution in the trans chamber. In addition, the invention relates to an apparatus for testing the function of a transmembrane molecule comprising the composite biomimetic membrane according to the invention and having the following features:

A two-cell chamber wherein each cell has an upper opening to allow access to the cell, and a membrane scaffold according to any one of the claims 1 to 6 comprising said composite biomimetic membrane, which provides a partition between the two cells to form a cis chamber and a trans chamber, a partial separation (7) in the cis chamber which extends from the top of said chamber to below said functional area thus forming a relatively narrow space with said scaffold (4), a porous support layer (3) which is a functional water barrier at atmospheric pressure opposite the partial separation (7), a first volume of aqueous buffer solution in the trans chamber opposite the partial separation (7) where said volume extends above said central area of said scaffold (4), a second volume of aqueous buffer solution in the cell having the partial separation (7) where said volume does not reach the lower level of said functional area of said scaffold (4), a spacer (5) is provided between said partial separation (7) and said scaffold (4), said spacer having an upper opening to allow insertion of a syringe. The apparatus may further include elastic seals (2, 6) that are inserted between parts 1 and 3, 4 and 5, 5 and 7, 7 and 8, 8 and 9, and between 9 and the annular sealing screw, said elastic seals being of a chemically resistant material, such as a fluoroelastomer, e.g. Viton®. The reference numbers are found in FIG. 12. In the apparatus an electrode may be inserted in each of said upper openings and in contact with said first and second “buffer” solutions.

In a preferred embodiment of the apparatus of the invention said said transmembrane molecule is alpha-hemolysine, and a further aspect of the invention is the use of the apparatus according for the testing of a compound having binding effect on alpha-hemolysine said testing comprising adding a solution of said compound to said cis chamber and measuring conductance through said electrodes.

### BRIEF DESCRIPTION OF THE DRAWINGS

FIG. 1 is a drawing showing an overview of geometries needed for theoretical bulge calculation. is an optical microscopy picture of the scaffold with apertures.

FIG. 2 is a SEM micrograph of an ETFE scaffold of the invention showing a close up on apertures turned 60o to show bulges in an array with 140 μm spacing. The rough surface is due to the gold which is sputtered on for better contrast.

FIG. 3 is a SEM photograph showing the central area with apertures of a scaffold according to the invention having 120 μm spacing and turned 45o.

FIG. 4 is a SEM photograph showing a section of a scaffold according to the invention having an aperture diameter of about 300 μm and a bulging aperture rim.

FIG. 5 shows a 5×5 array in rectangular design with spacing of 150 μm.

FIG. 6 shows SEM pictures of two scaffold arrays of the invention made in Tefzel 100LZ ETFE film (DuPont) with 140 μm spacing.

FIG. 7 is an SEM picture showing most parts of an entire scaffold of the invention having a central 20×20 aperture array in hexagonal design and with 150 μm spacing and an outer nonperforated area.

FIG. 8 is a graph showing a Dektak profilometer measurement of a scaffold having average aperture diameter of 84.6 μm.

FIG. 9 is a drawing showing the APM method of preparing a biomimetic membrane, e.g. a BLM membrane, in the apertures of the scaffold of the invention creating a composite biomimetic membrane. Shown is a sectioned schematic side view through the middle of an assembled two-cell Teflon chamber. In steps 1-3 the buffer level in the cis chamber is raised above the aperture, thus creating a lipid bilayer (red line, step 3) by the parallel raising of the DPhPC/decane layer (red square, step 1-3).

FIG. 10 shows schematically the Folded bilayers method according to Montal & Muller (1972, PNAS, 69:3561-3566).

FIG. 11 shows schematically the Painted bilayers method according to Muller & Rodin (1969, Cur. Top. Bioeng. 3:157-249).

FIG. 12 shows the movable inner parts of an embodiment of the two-cell Teflon chamber. The inner diameter of Viton seals and Teflon spacers is 8 mm. A thin layer of silicone grease (High Vacuum Grease, Dow Corning) is applied to the inner Viton seals prior to assembly. An annular sealing screw (not shown) secures sealing from the right end as shown by the arrow. It is possible to visually follow the formation of lipid membrane through the opening in the annular sealing screw.

FIG. 13 is a drawing showing various views of the solid, outer parts of an APM-1 chamber of the invention.

FIG. 14 is a drawing showing the T-ring.

FIG. 15 is a drawing showing the annular sealing screw.

FIG. 16 is a graph showing changes in conductance of a composite membrane after adding valinomycine and TEA.

FIG. 17 is a graph showing changes in conductance of a composite membrane after adding valinomycine and TEA in a different experiment.

FIG. 18 shows 4 diagrams of capacitance and the conductance for an experiment reported in example 10.

FIG. 19 shows 6 fluorescent images of traditional and airbrush pretreated multiple apertures.

FIG. 20 shows diagrams of the capacitance and the conductance for an airbrush pretreated membrane scaffold.

FIG. 21 discloses a diagram of conductance of a membrane incorporating valinomycin.

FIG. 22 shows 3 SEM images of the scaffold membrane used in example 10.

FIG. 23 shows in 4 sequences the formation of a membrane by the APM method.

FIG. 24 shows the hexagonal configuration of an aperture array of the invention.

FIG. 25 are Photomicrographs of composite biomimetic membranes made in a Fluon 50N scaffold material comprising BLMs in 8×8 arrays in the horizontal chamber setup, 300 micrometre diameter apertures and centre-to-centre distance of 400 micrometres, cf. FIG. 26. The figures show functional incorporation of alpha-hemolysin channels in composite biomimetic membrane array of the invention. 25A is a fluorescence image of an 8×8 BLM array using a 2.5× objective, 25B+C show a transmitted light image and the corresponding fluorescent image using a 10× objective, and 25D is a graph showing conductance in pA of said membrane array as a function of time.

FIG. 26 shows combined horizontal imaging and electrical voltage clamp chamber design.

### DETAILED DESCRIPTION OF THE INVENTION

One aspect of the invention relates to a membrane scaffold comprising a planar material having a hydrophobic surface (such as an ETFE film) and a central perforated area wherein a plurality of essentially circular apertures having smooth, bulging rims have been formed using a CO₂ laser ablation process. The membrane scaffold has preferably a thickness of from about 25 μm to about 200 μm. The rounded and bulging rims of the apertures in the membrane scaffold of the invention possess several advantages in contrast to apertures having blunt-edged rims, e.g. by/in stabilizing the membrane formed in the apertures against breakdown and in supporting a stable torus or annulus of fluid membrane forming composition, such as an amphiphilic lipid solution, for the sustainability of the fluid biomimetic membrane during evaporation of solvent. A toroidal membrane forming solution reservoir will act as a reservoir in equilibrium with the bilayer membrane allowing for exchange of material necessary for bilayer bulging (e.g. when under pressure) and self-repair.

### Definitions:

The term “Biomimetic membrane” as used herein is intended to cover planar molecular structures having an upper and a lower hydrophilic layer and an inner hydrophobic layer resembling the structure of a eukaryotic cell membrane.

“BLM” as used herein means Black Lipid Membrane or Bilayer Lipid Membrane. The term “aperture diameter” as used herein always refers to an average measured diameter of the apertures in the entire scaffold. The term “essentially circular” is used herein to characterize the cross sectional shape of the apertures in the scaffolds of the invention. It is believed that this shape is ideally circular for optimal support of a biomimetic membrane, such as a lipid bilayer. However, various approximately circular forms including ovals or ellipses and rounded tetragonal or box-like forms are intended to be included in the term.

“Buffer” is used herein to describe a solution comprising one or more electrolytes with or without buffering capacity.

“Smoothness” as used herein refers especially to the aperture rims that ideally do not have blunt edges or cracks.

The term “bulge” is used herein to denote the enlarged height of the apertures relative to the thickness of the film in which they are formed using the laser ablation process. Especially when using a CO₂ laser ablation to form the apertures some film material will accumulate along the rim to form the bulge. For the purposes of the invention the bulges have to be smooth and rounded and should not be too high. The geometry of the bulge is described in more detail below.

The term “torus” is used herein to describe a peripheral ring of multilayered amphiphilic lipid solution surrounding the central bilayer membrane formed in the aperture.

“APM” means Auto-Painted Membrane the formation of which is described in Example 2 below.

“Teflon” as used herein includes ETFE, polyethylene-tetraflouroethylene, and modifications and derivatives thereof; ECTFE, polyethylene-chlorotrifluoroethylene, and modifications and derivatives thereof; PTFE, Polytetrafluoro-ethylene and modifications and derivatives thereof; FEP, Fluorinated ethylene propylene and modifications and derivatives thereof. Teflon is used synonymous with flouropolymer. DPhPC means 1,2-diphytanoyl-sn-glycero-3-phosphocholine. EtOH means ethanol.

“ETFE” as used herein includes polyethylene-tetraflouroethylene, and modifications and derivatives thereof; as well as ECTFE, polyethylene-chlorotrifluoroethylene and modifications and derivatives thereof.

“BFS” means bilayer forming solution and is used herein interchangeably with the term “Membrane forming solution” and specifies a mixture of an amphiphilic substance with an apolar solvent to obtain a liquid solution suitable for forming membranes.

The terms “film” and “foil” are used interchangeably herein when describing the planar material used in fabricating the membrane scaffolds, and the term “elastic” is used to characterize sealing means that can be made of an elastomeric material or other rubber-like material.

CO₂-Laser: The process used in forming the apertures is preferably a laser ablation (laser photoablation), preferably using a CO₂ laser (e.g. Synrad, Inc. 4600 Campus Place Mukilteo, Wash. 98275 USA, Laser: 48-of the 48 series (50 W laser)) which will secure high reproducibility, well defined aperture diameters, and a high degree of aperture density in the planar scaffold material. In addition a laser ablation method can easily be upscaled. The membrane scaffold according to the invention is preferably prepared using an optically induced/stimulated thermal process, such as a CO₂ laser ablation, where said laser beam is preferably operated at a power of about 3 W to about 8 W or more. An advantage of using a thermal process is the partly melting of the material resulting in forming of the smooth rims without any sharp edges. Further advantages include low power consumption and that the laser itself having small dimensions is mountable on a stage together with other lasers for production of large scale scaffolds, e.g. in m² scale. The CO₂ laser emits infrared light with a wavelength of 10.6 μm in a continuous beam. The decomposition of the planar material takes place due to thermal processes only. When the beam hits the surface of the sample the polymer melts and parts are vaporized. The gas drives the melted polymer out of the void which results in a bulge around the edges of the structure. It is a fast and inexpensive method which is mainly used in direct writing. Every polymer with sufficient absorption in this region can be processed.

The CO₂ laser ablation is a mere thermal process. This means that parts of the planar material surrounding the aperture are influenced by the thermal process and bulges are left behind. The minimal structure size depends on the optical components used in the setup. For example with a lens with a focal length of 80 mm apertures of 116 μm were reported to be the minimum (Jensen, M. F., et al. 2003.—Microstructure Fabrication with a CO₂ Laser System: Characterization and Fabrication of Cavities Produced by Raster Scanning of the Laser Beam. Lab on a chip. 3 pp 302-307). Scaffold material The scaffold material is chosen to be hydrophobic, preferably having a contact angle of more than 90°, or preferably more than about 100° as measured between a droplet of de-ionised water and the hydrophobic material. The contact angle measurement is performed at 20° C. and atmospheric pressure using a contact angle goniometer. Suitable hydrophobic materials include films made of various crystalline or semicrystalline fluoropolymer materials (Teflon®) such as ETFE (ethylene Tefzel® ETFE, DuPont™), Fluon ETFE Film 50N (by Asahi Glass Company, Ltd.) and Norton ETFE, ECTFE (Saint-Gobain Performance Plastics Tygaflor Ltd.). These film materials are susceptible to the ablation process of the CO₂ laser. Crystalline polymers have a relatively sharp melting point where the crystalline lattice is destroyed which is characterized by the crystalline melting temperature Tm. It is desired that the scaffold material is able to absorb infrared light with a wavelength of 10.6 μm, and therefore a relatively low transmittance at this wave length is desirable. A preferred example of a suitable scaffold material is ethylene-tetrafluoroethylene (ETFE) which has an transmittance at 10.6 μm of 88.2%.

The planar hydrophobic material must be resistant towards the chemicals used in the process of forming the membranes in the apertures. The material must be able to withstand the complex cleaning steps used prior to establishing the biomimetic membrane, e.g. a lipid bilayer. The material needs to withstand, e.g., chloroform, hexane and DPhPC/decane (2.5 wt %). SEM pictures of the apertures were taken before and after this chemical treatment to provide the basis to compare any changes in aperture diameter as well as in the overall appearance of the structure. The chemical resistance tests have shown that the crystalline or semicrystalline Teflon materials, such as ETFE were sufficiently chemically stable. The experiments with the different chemicals did not show any damage on the ETFE scaffold apertures. A comparison between the aperture diameter before and after the treatment confirmed the results from the visual inspection.

TABLE 1
Properties of various Teflon materials

| | Poly- | Fluorinated | Ethylene- |
|---|---|---|---|
| | tetrafluoro- | ethylene | tetrafluoro- |
| | ethylene | propylene | ethylene |
| Property | (PTFE) | (FEP) | (ETFE) |
| Polymer | Thermo- | Thermo- | Thermo- |
| type | setting | setting | setting |
| Melt | 327° C. | 250- | 250- |
| temperature | | 280° C. | 270° C. |
| Transmittance | 97.5% | 97.5% | 88.2% |
| at 10.6 μm | | | |
| Contact | 106° | 105° | 105° |
| angle | | | |
| (water) | | | |
| Manufacturer | DuPont ™ | DuPont ™ | DuPont ™ |

### Scaffold Geometry

The membrane scaffold according to the invention has preferably a central functional area having a degree of perforation of about 20% to about 60% and more preferably from about 30 to about 50%. In addition, the membrane scaffold comprises a circumscribing area of unperforated film which is useful when sealing the scaffold into a tight chamber. In the membrane scaffold according to the invention the spacing between the apertures is preferably from about 150 μm to about 500 μm measured as the distance between aperture centres. The spacing is preferably from about 130% of the aperture diameter to about 500% of the aperture diameter. It has been found that this spacing will allow bulge formation of the aperture rims, which may further stabilize the membrane formation and/or longevity of the membranes. However, in some embodiments of the invention the interspace between neighbouring apertures is so reduced that two separate bulges cannot be formed. Instead they combine and build up one bulge ranging from the edge of one aperture to the neighbouring one with the highest point approximately in the middle of the interspace, cf. FIG. 2 and FIG. 3 showing a picture of a scaffold having 84 μm aperture diameter and 120 μm spacing where this phenomenon is visible.

In a specific embodiment of the invention the membrane scaffold has a central perforated area of about 3.1 mm×3.1 mm having 8×8 apertures (diameter 300 μm) and center to center distance of 400 μm in a rectangular arrangement where the scaffold was made from an ETFE film of 0.001 inch (25.4 μm) thickness (Tefzel 100 LZ, DuPont®).

The apertures are preferably of relatively smaller dimensions, such as about 80 to 200 μm, when the composite biomimetic membrane formed using the membrane scaffold is to be used for applications such as biosensors.

In the membrane scaffold according to the invention said planar material has typically a thickness of from about 25 μm to about 300 μm, where the thinner materials are suitable for apertures having the larger diameters, and the thicker materials are suitable for applications requiring applied pressure, such as filtration of water. The planar material having a hydrophobic surface is preferably an ETFE film having a contact angle of about 95°-106° and a thickness of between about 25 to 100 μm or more preferably of about 50 μm to about 60 μm.

### Theoretical Geometry Considerations

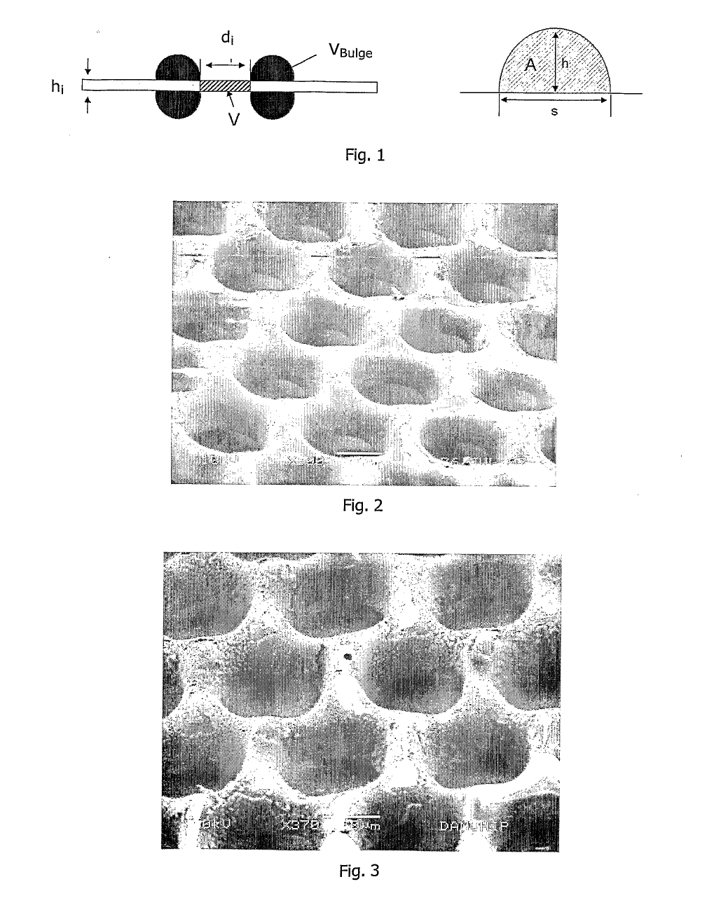

The material accumulations during the laser ablation process are of importance for the final shape of the membrane scaffolds. The rims of the apertures desirably are smooth and round and should not be too high to ensure stable lipid bilayer formation. Therefore, a model of the bulge was developed. With these equations and the parameters s and di measured on a SEM picture, the expected height could be determined. Several assumptions have been made to simplify the calculation. First of all, vaporization and increase of volume of the ablated polymer were neglected. Furthermore, it was expected that the bulges will have an elliptical vertical cross-sectional shape and that they are equal all around the aperture. FIG. 1 shows a cross-section of a perforation to the left and a cross section of a bulge to the right.

When “shooting” the aperture hole, all material which can be accumulate in a bulge must be displaced volume from the aperture. This means the displaced volume is:

$$V = π * \frac{{d}_{i}^{2}}{4} {h}_{i} \qquad ( 5.1 )$$

This volume is then deposited to form the bulge which surrounds the hole on both sides of the foil. It depends on the diameter of the hole di and the width s of the bulge.

V_{bulge}=A*l (5.2)

l=U=π*(dᵢ+s) (5.3)

The parameter l describes the perimeter of the circle on which outer side the maximum bulge height was expected. When looking at one side only, the value h is half the length of the major axis of the ellipse.

$$A = π * \frac{s}{2} * h \qquad ( 5.4 )$$

By measuring di and s for example on a SEM picture, the height of the bulge then arises from Eq.(5.1), Eq.(5.3) and Eq.(5.4) to be:

$$h = \frac{{d}_{i}^{2} * {h}_{i}}{2 * π * s * ( {d}_{i} + s )} \qquad ( 5.5 )$$

A calculation has been made to find the best suitable arrangement of apertures. As widely known from literature, the highest density which can be achieved is when using a hexagonal structure. This can also be applied to the membrane scaffold of the present invention. The area which is covered by this structure can be calculated by having the spacing between the holes a, the number of apertures within each row x and the number of rows y. This results in the length l and width w which can be calculated by:

$$\begin{matrix}l = a * x & ( 7.1 ) \\ w = h * ( y - 1 ) + a & ( 7.2 ) \\ h = \sqrt{3} * \frac{a}{2} & ( 7.3 )\end{matrix}$$

The final covered area then is:

A=l*w (7.4)

However, it has to be taken into account that x defines the maximum number of apertures in a row. In a maximum density hexagonal structure this number is different in the even and uneven numbers of rows. This calculation assumes that the used array starts and ends with an uneven row number which has one aperture more than an even one. When having an area which has to be covered with apertures this calculation has to be performed backwards. Then, the amount of apertures for an even row can be calculated by:

$$x = \frac{l}{a} \qquad ( 7.5 )$$

The number of rows is defined to be:

$$y = \frac{2 * ( w - a )}{\sqrt{3} * a} + 1 \qquad ( 7.6 )$$

The resulting values have to be rounded to fulfill the requirements of having integer values and an uneven number of rows. By taking the average diameter d of the apertures into account, the perforation level p can now be calculated by:

$$p = \frac{{A}_{holes}}{A} * 100 \% \qquad ( 7.7 )$$

Here Aholes defines the area where material was removed.

$${A}_{holes} = ( \frac{1}{4} * π * {d}^{2} ) * z \qquad ( 7.8 )$$

The value z is the overall number of apertures in the area Aholes. Table 2 lists the percentage of perforation for different spacings (center-to center distance) and an aperture diameter of 89 μm in average when filling an area of approximately 2×2 cm.

TABLE 2
Theoretical level of perforation which can be achieved
by hexagonal arrangement of the apertures in a 2 ×
2 cm sample with an average aperture diameter of 89 μm

| | | | number of | |
|---|---|---|---|---|
| spacing a | x | Y | apertures z | perforation level p |
| 150 μm | 133 | 153 | 20,273 | 31% |
| 140 μm | 143 | 163 | 23,228 | 36% |
| 130 μm | 154 | 177 | 27,170 | 41% |
| 120 μm | 167 | 191 | 31,802 | 49% |

TABLE 3
Perforation level, calculated for rectangular arrangement of the
apertures in a sample with an average aperture diameter of 300 μm

| | | number of apertures | | | |
|---|---|---|---|---|---|
| | | (same in x and y) | | | |
| | | 8 | 10 | 100 | 1000 |
| level of | c-c | 21.19% | 20.87% | 19.75% | 19.65% |
| perforation | distance = 600 μm | | | | |
| level of | c-c | 43.63% | 43.74% | 44.13% | 44.17% |
| perforation | distance = 400 μm | | | | |
| level of | c-c | 54.92% | 55.46% | 57.47% | 57.68% |
| perforation | distance = 350 μm | | | | |

TABLE 4
Perforation level, calculated for hexagonal arrangement of the
apertures in a sample with an average aperture diameter of 300 μm

| | | number of apertures | | | |
|---|---|---|---|---|---|
| | | (different in x and y) | | | |
| | | 8 | 10 | 100 | 1000 |
| level of | c-c | 22.73% | 22.71% | 22.67% | 22.67% |
| perforation | distance = 600 μm | | | | |
| level of | c-c | 46.78% | 47.53% | 50.63% | 50.97% |
| perforation | distance = 400 μm | | | | |
| level of | c-c | 58.87% | 60.24% | 65.92% | 66.56% |
| perforation | distance = 350 μm | | | | |

TABLE 5
Perforation level. Calculation for 2 × 2 cm scaffold, cf. FIG. 24

| Rectangular arrangement; Aperture diameter | 300 | μm |
|---|---|---|
| 2 cm² area; c-c distance = | 400 | μm |
| number of apertures (same in x and y) | 50² | |
| level of perforation | 46.01% | |
| Hexagonal arrangement; Aperture diameter | 300 | μm |
| 2 cm² area; c-c distance = | 400 | μm (x) |

| | |
|---|---|
| number of apertures in x | 50 |
| number of rows (y = 347 μm) | 57 |
| number of holes | 2822 |
| level of perforation | 50.80% |

With these densely packed structures and the huge amount of apertures another parameter came into focus—the Off Vector Delay (OVD). By reducing this parameter to a value as low as possible, precious time during production could be saved. For example by reducing OVD from 600 μs to 1 μs with a production volume of 20,273 apertures the production time can be shortened by 12 s. This is an advantage regarding the further up-scaling of the membrane scaffold fabrication where it can result in reduction of considerable production time. However, this reduction of OVD could change the overall parameters because the material would have less time to cool down between production steps.

### Anisotropy of the Teflon Film Used in Preparation of the Membrane Scaffold

The used ETFE film is available on 25 m² rolls which were processed by conventional melt-extrusion techniques (DuPont™). There are two main directions which are significant in this process. The machine direction (MD) which is oriented along the length of the sheet and perpendicular the transverse direction (TD) which defines the characteristics of the film across the width of the film. During the process of being stretched and pressed, polymer chains tend to align in a parallel form. By the immediate following cooling process this alignment is frozen in. Orientation of the material leads to higher strength in this direction than at right angles. This characteristic of having an orientation should be taken into account when forming the aperture array. It was found for the Tefzel 100LZ film that while lowering the distance between center-to-center of the apertures, the shape of the holes became more and more elliptical. Thus, there is a dependency on the material's orientation for the production of closely spaced arrays. Furthermore, it has to be noted that the apertures produced with the MD were not completely round when having a low distance. They tended to be more hexagonal and formed an array which can be compared to a honeycomb. The tendency to form elliptical holes can be explained by the orientation of the molecules within the polymer. They tended to align parallel after the film extrusion. When shooting an aperture, high thermal energy is induced which breaks bonds between the polymer chains. In the immediate focal spot of the laser this causes melting and vaporization of the material. The surroundings of the aperture was also affected by this thermal energy, plus the energy induced from neighbouring apertures. When producing perpendicular to MD the bonds between the parallel chains may break and cracks develop. This could explain the elliptical apertures. However, when fabricating with MD this effect did not occur because within a very short time (˜1 μs) a neighbouring aperture was produced and additional material was placed on the partition between the two apertures. The fabrication of several closely spaced apertures and the later increase of the perforation level showed that not only the settings of the laser influenced the quality of the aperture. It has been found that the characteristics of the material are important as well. Primarily, this applied for the alignment of the polymer chains due to the fabrication process of the foil. Tests showed that the direction of aperture fabrication is preferably parallel with the machine direction of the ETFE film. This resulted in dense arrays with a honey comb like structure. However, this only applied for the fabrication of the smallest apertures in dense arrays. When producing the apertures with a larger diameter and a larger spacing the optimal production direction changes to be perpendicular to MD. Here, structuring in the machine direction resulted in more oval apertures. Compared to the single aperture approach, the fabrication settings of the CO₂ laser itself had to be modified when making arrays. Significant here was the OVD which could be reduced from 600 μs to 1 μs. This change was possible due to the closely arranged apertures. The OVD vs. spacing test revealed that slight changes in diameter and shape were possible with changing time. A higher number of apertures will lead to more time between the fabrications of rows and thus every single aperture will be influenced only by its two immediate neighbours from the same row. Therefore, and considering the time of the fabrication process a minimum OVD was preferred. The second important parameter was the spot lase duration. This value had to be changed within the same structure. Outer apertures (mostly the ones starting a new row) required a higher value than those following. That proved the influence of the heat, coupled in by the ablation process. The thermal energy lowers the threshold of the melting and vaporizing of the ETFE. That made it possible to produce apertures in a shorter time. The first aperture of an array always had to be 5 ms which is higher than the rest but lower than needed to produce a single aperture. Depending on the distance between the apertures, this time could be further reduced down to 4 ms. This effect was also observable when measuring the diameter, for example in the OVD vs. spacing test. Apertures closer to the middle of the array were always bigger than the ones on the outside. Therefore, when determining the average diameter for a structure, only apertures from inside the array were measured. Different center to center distances resulted in different average diameters. This was caused by the thermal energy from the production of previous apertures in the array. As mentioned before, this also led to a decrease in spot lase duration in denser arrays which in turn led to a smaller diameter for shorter spacings.

### Channel Forming Molecule

The membranes formed in the scaffolds of the invention readily incorporate channel forming molecule, e.g. a peptide ionophore such as valinomycin that exists in natural lipid bilayer membranes, cf. Example 8 below or aquaporins, such as bovine AQP-1 and plant plasma membrane aquaporins of the PIP subfamily, e.g. SoPIP2;1. The channel forming molecule may be incorporated in the membrane by direct incorporation at the membrane formation step, where the aquaporin proteins are first incorporated in a suitable hydrophobic spreading solution. The spreading solution can be prepared from aqueous SoPIP2;1 extract emulsified with the lipid, e.g. DPhPC in hydrophobic solvent, e.g. n-decane, cf. Walton et al., Anal. Chem. 2004, 76, 2261-2265. SoPIP2;1 can be obtained in the form of a heterologously expressed protein, cf. Kukulski W et al. Journal of molecular biology (2005), 350(4), 611-6. Thus, said channel forming molecules are preferably selected from the group consisting of ion channel molecules, such as valinomycin and gramicidin monomers and dimers, transmembrane proteins such as porins e.g. outer membrane protein OmpG, phosphoporin PhoE and aquaporin water channels, connexins e.g. Cx26, Cx30, Cx32, Cx36, Cx40, Cx43, etc., transporters such as light absorption-driven transporters e.g. bacteriorhodopsin-like proteins including rhodopsin and opsin, light harvesting complexes from bacteria, etc., ABC (ATP-binding cassette) transporters facilitating transport of small solutes and molecules such as ions, salts, antibiotics, etc. in a type-dependent manner, ABC subclass A transporting cholesterol, sphingolipids and phospholipids in a type dependent manner (Piehler et al. 2007, Tidsskr. Nor. Laegeforen., Vol. 127, No. 22. Review), Multidrug resistance pumps transporting antibiotics (Alekshun and Levy 2007 Cell Vol. 128), lead and mercury ion pums (e.g. CadA, ZntA and MerC, Rensing et al. 1998, J. Biol. Chem., Vol. 273, No. 49; Sasaki et al. 2005, Biosci., Biotechnol. Biochem. Vol. 69, No. 7), cation diffusion facilitator (CDF) protein family transporting heavy metal ions such as zinc, cobalt, cadmium (e.g. CzcD, Anton et al. 1999, J. Bacteriol., Vol. 181, No. 22), receptors such as neurotransmitter receptors e.g. GABA transporters, monoamine transporters, glutamate transporters, etc., CD-receptors such as CD-95, a receptor for serum Fas ligand, which is a serological marker for different disease states in humans including certain hormone sensitive cancer forms e.g. breast carcinoma, chemosensitivity in colorectal cancer, disease activity and infection states such as malaria or the asymptomatic stage of human immunodeficiency virus infection, etc. (Kuwano et al. 2002, Respirology, Vol. 7 Issue 1.; Kern et al. 2000 Infect. Immun. 68(5); Bahr et al. 1997, Blood, Vol. 90, No. 2), transmembrane CC chemokine receptor for which macrophage-derived chemokine (MDC) is a ligand and whose serum levels are elevated in atopic dermatitis differentiable from psoriasis activity (Kakinuma et al. 2002, Clin. Exp. Immunol., Vol. 127), CXC chemokine receptors, interleukin receptors, olfactory receptors and receptor tyrosine kinases e.g. the maturation-mediating receptor tyrosine kinase Tie-2 whose ligands include soluble angiopoietin-2, which has been identified as a biological marker in serum for non-small cell lung cancer with distant metastasis (Park et al. 2007, Chest., Vol. 132, Fiedler et al. 2003, J. Biol. Chem., Vol. 278, Issue 3). A useful channel protein is POR1 which forms a channel through the cell membrane that allows diffusion of small hydrophilic molecules. The channel adopts an open conformation at low or zero membrane potential and a closed conformation at potentials above 30-40 mV. The open state has a weak anion selectivity whereas the closed state is cation-selective. It is the major permeability factor of the mitochondrial outer membrane. Other interesting membrane proteins include the bacterial DsbB electron donor and the cystic fibrosis transmembrane regulator (CFTR) which functions as a cAMP-activated chloride channel and also regulates a separate protein, the outwardly rectifying chloride channel (ORCC). Other useful channel forming molecules identified from several ORFs are listed in Burri et al. 2006 FEBS Journal Vol. 273. Also preferred is the heptameric channel forming protein alpha-hemolysin.

### Biomimetic Membrane

In the biomimetic membrane of the invention said lipid is preferably selected from amphiphilic lipids, such as DPhPC or DPPC. WO2006122566, the contents of which are incorporated herein by reference, discloses useful amphiphilic compounds and lipids for reconstitution of aquaporins and formation of lipid bilayers or biomimetic membranes, cf. Table 1 therein. In addition, DPhPC (diphytanoylphosphatidylcholine, Avanti Polar Lipids, Alabaster) and DPPC, SOPC, DOPC, asolecthin, E. coli total lipid extract, SOPE, DOPE, DOPS and derivatives and mixtures thereof are preferred lipids for use in the biomimetic membranes of the present invention. The lipid is preferably dissolved at a concentration of from about 2 mg/mL to about 100 mg/mL in an apolar solvent, such as hexane, octane, decane, tetradecan, hexadecane, etc., in order to obtain a suitably fluid membrane forming composition. Preferred solvents are n-decane, n-tetradecane, and n-hexadecane. Without being bound by any theory it is assumed that the most suitable solvents possess a carbon chain which is approximately of the same length scale as the acyl carbon chains of the amphiphilic lipids. Said lipid bilayer may further comprise a bilayer stabilising amount of one or more stabilizing substances, such as cholesterol, dextran, or a monosaccharide, a sugar alcohol, a disaccharide, a trisaccharide, an oligosaccharide, a polysaccharide as disclosed in US 2005/0048648.

Useful methods of preparing lipid bilayer membranes in the apertures of the scaffold of the invention to form composite biomimetic membranes are described in WO2006122566 the contents of which is incorporated herein by reference. A preferred method herein is the APM method described in Example 10 below.

In some embodiments of the invention the biomimetic membranes can be formed in the scaffold apertures from solutions of amphiphilic block copolymer simulating a natural environment. Functional membrane molecules can be incorporated in this type of biomimetic membrane. One method of forming a biocompatible membrane, which is preferred for use with block copolymer-based membrane, is as follows: Form a solution of block copolymer in solvent (BC solution). The solution can be a mixture of two or more block copolymers. The solution preferably contains 1 to 90% w/v copolymer, more preferably 2 to 20%, or yet more preferably 20 to 10%, such as 7%. Make a solution of channel forming molecule such as aquaporin in the prepared BC solution, preferably by adding 1.0 to 50.0 mg/mL of the preferred aquaporin, more preferably 1.0 to 10.0 mg/mL. Drop a small volume (e.g., 4 microliter) aquaporin/BC solution onto each aperture or each of a subset of apertures, and allow to dry, thereby removing the solvent. Repeat this step as needed to cover all apertures. The solvent is selected to be miscible with both the water component used in the process and the B component of the block copolymer. Appropriate solvents are believed to include methanol, ethanol, 2-propanol, 1-propanol, tetrahydrofuran, 1,4-dioxane, solvent mixtures that can include more apolar solvents such as dichloromethane so long as the mixture has the appropriate miscibility, and the like. (Solvent components that have any tendency to form proteindestructive contaminants such as peroxides can be appropriately purified and handled.) Solvent typically comprises 10% v/v or more of the applied aquaporin/BC solution, preferably 20% or more, and usefully 30% or more. The above-described method of introducing aquaporin or other desirable membrane channels as described herein to a solution containing nonaqueous solvent(s) in the presence of block copolymers serves to stabilize the function of active channels, such as aquaporins. The non-aqueous components can comprise all of the solvent. The mixtures of block copolymers can be mixtures of two or more of the following classes, where the separate components can be of the same class but with a different distribution of polymer blocks: Polymer Source triblock copolymers E/EP/E, of poly(ethylene)(E) and poly(ethylene-propylene)(EP) Triblock copolyampholytes. Among (N,N dimethylamino)isoprene, such polymers are 15 Ai14S63A23, Ai31S23A46, Ai42S23A35, styrene, and methacrylic acid Ai56S23A21, Ai57S11A32. Styrene-ethylene/butylene-styrene (KRATON) G 1650, a 29% styrene, 8000 solution triblock copolymer viscosity (25 wt-% polymer), 100% triblock styrene-ethylene/butylene-styrene (S-EB-S) block copolymer; (KRATON) G 1652, a 29% styrene, 1350 solution viscosity (25 wt-% 20 polymer), 100% triblock S-EB-S block copolymer; (KRATON) G 1657, a 4200 solution viscosity (25 wt-% polymer), 35% diblock S-EB-S block copolymer; all available from the Shell Chemical Company. Such block copolymers include the styrene-ethylene/propylene (S-EP) types and are commercially available under the tradenames (KRATON) G 1726, a 28% styrene, 200 solution viscosity (25 wt-% polymer), 70% diblock S-EB-S block copolymer; (KRATON) G-1701X a 37% styrene, >50,000 solution viscosity, 100% diblock S-EP block copolymer; and (KRATON) G-1702X, a 28% styrene, >50,000 solution viscosity, 100% diblock S-EP block copolmyer. 30 Siloxane triblock copolymer PDMS-b-PCPMS-b-PDMSs (PDMS=polydimethylsiloxane, PCPMS=poly(3-cyanopropylmethylsiloxane) can be prepared through kinetically controlled polymerization of hexamethylcyclotrisiloxane initiated by lithium silanolate end-capped PCPMS macroinitiators. The macroinitiators can be prepared by equilibrating mixtures of 3-cyanopropylmethylcyclo-siloxanes (DxCN) and dilithium diphenylsilanediolate (DLDPS). DxCNs can be synthesized by hydrolysis of 3-cyanopropylmethyldichlorosilane, followed by cyclization and equilibration of the resultant hydrolysates. DLDPS can be prepared by deprotonation of diphenylsilanediol with diphenylmethyllithium. Mixtures of DxCN and DLDPS can be equilibrated at 100 [deg.] C. within 5-10 hours. By controlling the DxCN-to-DLDPS ratio, macroinitiators of different molecular weights are obtained. The major cyclics in the macroinitiator equilibrate are tetramer (8.6+−0.7 wt %), pentamer (6.3+−0.8 wt %) and hexamer (2.1+−0.5 wt %). 2.5 k-10 2.5 k-2.5 k, 4 k-4 k-4 k, and 8 k-8 k-8 k triblock copolymers have been characterized. These triblock copolymers are transparent, microphase separated and highly viscous liquids. PEO-PDMS-PEO triblock Formed from Polyethylene oxide (PEO) and poly-copolymer dimethyl siloxane (PDMS). Functionalized poly(2-Angew. Chem. Int. Ed. 39: 4599-4602, 2000; Langmuir methyloxazoline)-block-16: 15 1035-1041, 2000. These A-B-Apolymers include poly(dimethylsiloxane)-blockversions in which the A components have MW of poly(2-methyloxazoline) triblock approximately 2 kd, and the B component of copolymer approximately 5 kd, and (b) the A components have MW of approximately 1 kd, and the B component of approximately 2 kd Poly(d/1-lactide)(“PLA”)-PEG-PLA triblock copolymer Poly(styrene-b-butadiene-b-styrene) triblock copolymer Poly(ethylene Such polymers included Pluronic F127, Pluronic P105, or oxide)/poly(propylene oxide) Pluronic L44 from BASF (Performance Chemicals). Triblock copolymers PDMS-PCPMS-PDMS A series of epoxy and vinyl endcapped polysiloxane (polydimethylsiloxane-triblock copolymers with systematically varied molecular polycyanopropylmethylsiloxane) weights can be synthesized via anionic polymerization using LiOH as an initiator. Polydiene-polystyrenepolydiene available as Protolyte A700 from DAIS-Analytic, Odessa, Fla. Azofunctional styrene-butadiene-HEMA triblock copolymer Amphiphilic triblock copolymer carrying polymerizable end groups Syndiotactic polymethylmethacrylate (sPMMA)-polybutadiene (PBD)-sPMMA triblock copolymer Tertiary amine methacrylate triblock Biodegradable PLGA-b-PEO-b-PLGA triblock copolymer, Polylactide-b-polyisoprene-b-polylactide triblock copolymer, Poly(isoprene-blockstyrene-block-dimethylsiloxane) triblock copolymer, Poly(ethylene oxide)-block-polystyrene-block-poly(ethylene oxide) triblock copolymer, Poly(ethylene oxide)-poly(THF)-poly(ethylene oxide) triblock copolymer Ethylene oxide triblock Poly E-caprolactone Birmingham Polymers, Birmingham, Ala. Poly(DL-lactide-coglycolide) Birmingham Polymers, Poly(DL-lactide) Birmingham Polymers, Poly(L-lactide) Birmingham Polymers, Poly(glycolide) Birmingham Polymers, Poly(DL-lactide-co-caprolactone) Birmingham Polymers, Styrene-Isoprene-styrene triblock Japan Synthetic Rubber Co., Tokyo, Japan; MW=140 kg/mol; copolymer Block ratio of PS/PI=15/85. PMOXA(y)-PDMS(x)-PMOXA (y1) which is a poly(2-methyloxazoline)-block-poly(dimethylsiloxane)-block-poly(2-methyloxazoline) which may be symmetric (y=y1) or assymmetric; PMMA-b-PIB-b-PMMA Poly(methyl methacrylate) (PMMA) and polyisobutylene (PIB). PLGA-PEO-PLGA triblock Polymers of poly(DL-lactic acid-co-glycolic acid) copolymer (PLGA) and PEO. Sulfonated styrene/ethylene-butylene/styrene (S-SEBS) triblock copolymer proton conducting membrane Poly(l-lactide)-block-poly(ethylene oxide)-block-poly(1-lactide) triblock copolymer Poly-ester-ester-ester triblock copolymer PLA/PEO/PLA triblock copolymer The synthesis of the triblock copolymers can be prepared by ring-opening polymerization of DL-lactide or e-caprolactone in the presence of poly(ethylene glycol), using no-toxic Zn metal or calcium hydride as co-initiator instead of the stannous octoate. The composition of the copolymers can be varied by adjusting the polyester/polyether ratio. The above polymers can be used in mixtures of two or more of polymers in the same or different class. For example, in two polymer mixtures measured in weight percent of the first polymer, such mixtures can comprise 10-15%, 15-20%, 20-25%, 25-30%, 30-35%, 35-40%, 40-45% or 45-50%. Or, for example where three polymers are used: the first can comprise 10-15%, 15-20%, 20-25%, 25-30%, 30-35%, 35-40%, 40-45% or 45-50% of the whole of the polymer components, and the second can 10-15%, 15-20%, 20-25%, 25-30%, 30-35%, 35-40%, 40-45% or 45-50% of the remainder. Block co-polymers can be custom synthesized and obtained, e.g. from the following
http://www.encapson.eu/index.php?option=com_content&task=view&id=17&Itemid=32
http://www.polymer.de/services/custom-synthesis/#
http://www.pkasynthesis.com/
http://www.akinainc.com/
http://www.polysciences.com/

### Application of Membrane Scaffold

The invention relates in a further aspect to a filtration device for filtering essentially pure water comprising a composite biomimetic membrane comprising aquaporin water channels as described above. The advantages of using the composite membrane in said filtering device is closely related to the possibility of up-scaling the functional membrane area by the manufacturing of large, flexible, and relatively thin sheets having a large multitude of discrete membrane units. In addition, the composite membrane ensures that filtering ability is maintained even though one or more discrete membrane units have failed. This situation may especially apply to a filtration device having multi layer stacking of the individual composite membranes or 2D-aperture-arrays. The final dimensions of the stacked composite membranes will depend on overall robustness and on intrinsic permeability of the chosen membrane material/membrane composition.

Examples of how functional aquaporins can be incorporated into a water membrane have been described, however the present invention is not limited by these examples. The present invention relates to any composite biomimetic water membrane comprising a membrane scaffold as described herein with a biomimetic membrane comprising functional (channel) molecules reconstituted in its apertures. Other useful applications of said composite membrane include biosensor applications, such as a transmembrane protein functioning as receptor or channel, labeled with a fluorophore to make a protein-based biosensor sensitive to ligands, solutes or small molecules. Said biosensors incorporated into bimimetic membranes can be used for ligand-receptor interactions used in high throughput screening assays for diagnostic or prognostic purposes prepared in 96-multi well plates, lab-on-a-chip devices or build into point-of-care measuring devices, or serve as quantitative measuring devices of solutes or small molecules such as heavy metal ions e.g. cadmium, copper, lead, etc., or antibiotics and other polluting agents for quantitative on-the-spot water analysis, or blood analysis of animals and humans.

### Membrane Formation

Furthermore, the invention relates to a novel method of forming auto-painted membranes (APM) in said scaffold to prepare a composite biomimetic memrane, and a chamber for the preparation and holding of said composite biomimetic membrane. Surprisingly, the inventors have found that the principle of the Auto-Painted membrane (APM) technique which uses a narrow reservoir of a concentrated, limited volume of amphiphilic membrane forming solution (e.g. DPhPC lipid mixed with an apolar solvent, e.g. a hydrocarbon solvent) in direct connection with a buffer volume on the front side (cis chamber) of the vertically positioned scaffold/partition is able to facilitate preparation of a composite biomimetic membrane. When raising said buffer solution the amphiphilic membrane forming solution will be raised completely past the scaffold (Teflon partition) and in the process be deposited into the multiple apertures, which have been pre-painted with a solution of amphiphilic substance in an apolar solvent, to create a composite membrane in said scaffold apertures. This method involves spraying the membrane scaffold with a solution of amphiphilic lipid in a hydrocarbon solvent prior to step a) above. The amphiphilic lipid is dissolved at a concentration of from about 10 mg/mL to about 100 mg/mL in an apolar solvent. Preferably the lipid is DPhPC and the apolar solvent is selected from the group comprising hexane, octane, decane, and hexadecane.

The invention further relates to the use of a composite biomimetic membrane of the invention comprising aquaporin water channels in pressure retarded osmosis for the production of salinity power, or the use of a composite biomimetic membrane comprising aquaglyceroporin water channels in pressure retarded osmosis for the extraction of salinity power.

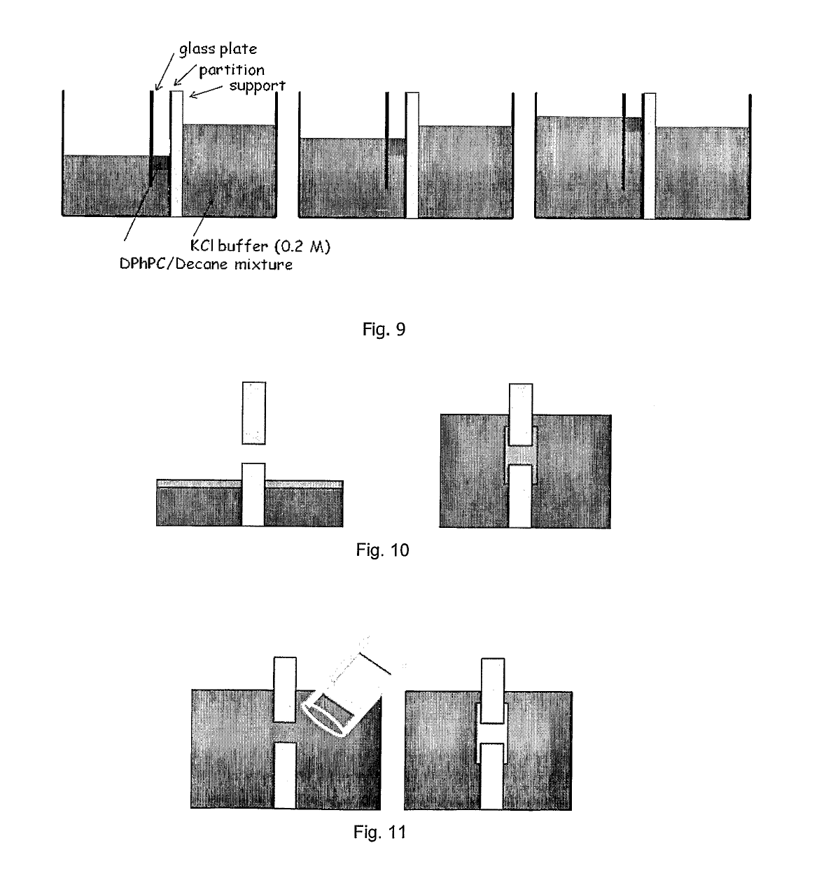

The hydrophobic nature of the scaffold surface ensures deposition of the apolar membrane forming solution into said multiple apertures. An optional feature of the APM method is that the composite membrane is supported and stabilized on the back side (trans chamber) by a preferably hydrophilic, porous support material that allows fluid connection between the membrane and the buffer solution in the trans chamber. In the APM-1 setting which is shown in FIG. 9 the 15 trans buffer level is just above the central perforated area of the scaffold where a negligible hydrostatic pressure will not result in flow of solution through the apertures. One advantage of the APM technique as compared to the folding and painting methods described in the art is the ease of up-scalability to create membranes in multi-aperture partitions without loss of reproducibility.

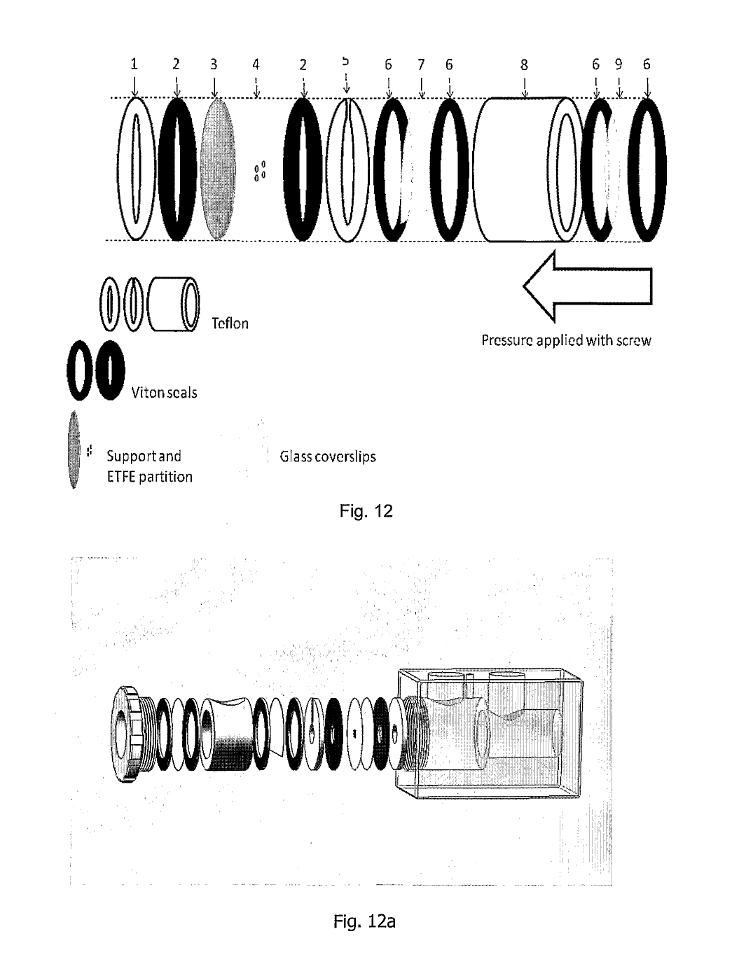

A general method of preparing a composite biomimetic membrane according to the invention comprises the steps of (reference numbers refer to FIG. 12):
a) providing a two-cell chamber wherein each cell has an opening allowing for access to the cell, and a membrane scaffold according to any one of the claims 1 to 6, which provides a partition between the two cells to form a cis chamber and a trans chamber;
b) providing a porous support which is a functional water barrier at atmospheric pressure;
c) providing a first volume of aqueous buffer solution in the trans chamber opposite the partial separation where said volume covers said central area of said scaffold;
d) providing a second volume of aqueous buffer solution in the cis cell opposite the partial separation where said volume covers said central area of said scaffold;
e) providing means to perfuse a volume (bolus) of membrane forming solution in the trans chamber thereby impregnating said functional area resulting in fluid membranes in said area; and
f) adding an extra volume of said aqueous buffer into either chamber to remove bolus leaving membranes in said functional area facing the trans and cis cell aqueous buffer.

The method described above is suitable for fabrication of composite biomimetic membranes in both the horizontal and in the vertical position and any position therein between

In another method of the invention a composite biomimetic membrane is prepared following the steps of (reference numbers refer to FIG. 12):
a) providing a two-cell chamber wherein each cell has an upper opening to allow access to the cell, and a membrane scaffold according to any one of the claims 1 to 6, which provides a partition between the two cells to form a cis chamber and a trans chamber,
b) providing a partial separation (7) in the cis chamber which extends from the top of said chamber to below said functional area thus forming a relatively narrow space with said scaffold (4),
c) providing a porous support (3) which is a functional water barrier at atmospheric pressure opposite the partial separation (7),
d) providing a first volume of aqueous buffer solution in the trans chamber opposite the partial separation (7) where said volume extends above said central area of said scaffold (4),
e) providing a second volume of aqueous buffer solution in the cell having the partial separation (7) where said volume does not reach the lower level of said functional area of said scaffold (4),
f) providing a volume of membrane forming solution in the space between the partial separation (7) and the scaffold (4), and
g) adding an extra volume of said aqueous buffer into said cis chamber to raise the buffer level above said functional area thereby raising the membrane forming solution completely past said apertures to form a fluid membrane therein.

A spacer (5) may be provided between said partial separation (7) and said scaffold (4), said spacer having an upper opening to allow insertion of a syringe; and elastic seals (2, 6) may be inserted between parts 1 and 3, 4 and 5, 5 and 7, 7 and 8, 8 and 9, and between 9 and the annular sealing screw, said elastic seals being of a chemically resistant material, such as a fluoroelastomer, e.g. Viton®.

The invention further relates to an apparatus for testing the function of a transmembrane molecule comprising the composite biomimetic membrane of the invention and having the following features:

A two-cell chamber wherein each cell has an upper opening to allow access to the cell, and a membrane scaffold of the invention comprising said composite biomimetic membrane, which provides a partition between the two cells to form a cis chamber and a trans chamber, a partial separation (7) in the cis chamber which extends from the top of said chamber to below said functional area thus forming a relatively narrow space with said scaffold (4), a porous support layer (3) which is a functional water barrier at atmospheric pressure opposite the partial separation (7), a first volume of aqueous buffer solution in the trans chamber opposite the partial separation (7) where said volume extends above said central area of said scaffold (4), a second volume of aqueous buffer solution in the cell having the partial separation (7) where said volume does not reach the lower level of said functional area of said scaffold (4), a spacer (5) is provided between said partial separation (7) and said scaffold (4), said spacer having an upper opening to allow insertion of a syringe.

Elastic seals (2, 6) may be inserted between parts 1 and 3, 4 and 5, 5 and 7, 7 and 8, 8 and 9, and between 9 and the annular sealing screw, said elastic seals being of a chemically resistant material, such as a fluoroelastomer, e.g. Viton®; and an electrode may be inserted in each of said upper openings and in contact with said first and second “buffer” solutions.

The apparatus described above may in an embodiment comprise a plurality of alpha-hemolysine oligomers incorporated in the biomimetic bilayer membrane, which enables the use of said apparatus for the testing of a compound having binding effect on alpha-hemolysine said testing comprising adding a solution of said compound to said cis chamber and measuring conductance through said electrodes. Positive reference measurements may be obtained in advance following addition of an inhibitor of alpha-hemolysine, e.g. beta-cyclodextrin and measuring the conductance.

Additional aspects of the invention relate to composite biomimetic membranes comprising aquaporins useful in the purification of a water source, or which can be used for pressure retarded osmosis (PRO), and in another aspect the present invention relates to the implementation of said membrane in a PRO system used in the production of salinity power, such as is described in WO/2007/033675.

### Examples

### Example 1

### Array Fabrication

To have an efficient membrane scaffold for, e.g. a filter membrane, the perforation level has to be as high as possible. Interactions from the production of neighbouring apertures in dense arrays influences the fabrication process when working with a CO₂ laser could be predicted. Due to be a thermal process, heat is coupled in the material each time the beam hits the surface of the film. This could lead to a lowering of the threshold where material is evaporated and thus result in bigger apertures in the middle of the array. Furthermore, when getting closer together, the bulges around the apertures could accumulate and so get higher in arrays than with single apertures. To investigate to what extent this may be the case, different arrays with different distances between the apertures and different parameters had to be designed and tested. To start the investigation and production of a highly perforated membrane, a simple 5×5 array of apertures was designed. It consisted of 5 rows of 5 apertures which were equally spaced. The term spacing refers to the distance between the centers of two neighbouring apertures. It was set to values of 500, 200 and 150 μm. With an average diameter of 96 μm this leaves an interspace of 404, 104 and 54 μm respectively between the apertures. The production parameters of the single aperture approach were transferred to the array—namely a power of 0.4 W and a spot lase duration (SLD) of 6 ms (see above). Furthermore, an Off Vector Delay of 600 μs was chosen to avoid tail formation like seen during the experiments with single apertures. The results confirmed the prediction of interactions between the apertures when coming closer together. With 500 μm spacing every aperture has an individual bulge, but at 200 μm and below the bulges start to touch each other. This can be explained by the thermal characteristic of the ablation by CO₂ laser. When the beam hits the surface of the material it melts. In addition, some parts are evaporated and the resulting gas ejects the melted material from the aperture. This is intensified by the heat which is coupled in and which makes the material softer and thus easier to deform. A series of experiments was set up with the previous design of a 5×5 array. This time the spacing was ranging from 250 down to 150 μm. Several experiments with changing laser powers and spot lase durations were performed. The results were investigated by optical microscopy. Here, the main criteria were the equality and the diameter of the apertures. The diameter was estimated with the help of a ruler integrated in the microscope's eyepiece. At the end of this test sequence the SLD could be reduced by 1 to 2 ms. It became obvious that it has to be changed within the grid. In the middle of the array the SLD could be lower than on the starting aperture to achieve the same results. This is again linked to the heat induced by the production. Since the apertures were close together and the time between production steps was short (600 μs), the material had no time to cool down. Due to the so preheated substrate less energy was needed to reach the melting point and the threshold of evaporation respectively. The optimization of the SLD resulted in an optimised array with smooth and almost round apertures. Furthermore, a decrease in the average aperture diameter could be observed. When producing single apertures the average diameter was ˜96 μm but in the array of 5×5 apertures with a spacing of 150 μm and a reduced SLD this value decreased by 7% to ˜89 μm. This is again connected to the heat induced by the production of neighboring apertures. As found when reducing SLD the diameter of the aperture decreased as well, cf. FIG. 5 which shows a 5×5 rectangular array with spacing of 150 μm. FIG. 6 shows SEM pictures of two scaffold arrays of the invention made in Tefzel 100LZ ETFE film (DuPont) with 140 μm spacing; the structure with the higher OVD (right side) has more circular holes whereas the one on the left side a more honeycomb like pattern.

### Example 2

### Geometrical Examination of Arrays

After having optimized the main production parameters, samples with spacing ranging from 150 μm to 120 μm were produced and examined to characterize the arrays and the apertures geometrically. It was found that the apertures at a center to center distance of 150 μm were completely round, however, their shape changed when decreasing the spacing. The cause was that every new aperture influenced its neighbours more and more with decreasing distance. The thermal energy induced by the laser melted and evaporated the material. Evaporating material built up a pressure which ejected melted parts but also pushed the softened boundaries. By having hexagonal arranged apertures this resulted in the formation of box like or even hexagonal shaped apertures, cf. FIG. 6. Using the previous model an estimation of the bulge height could be made. The highest bulge will form at the spot where two apertures get closest to each other. However, it has to be noted that this time no single hole was investigated but two or more. Therefore, the displaced volume of two holes is accumulated at the interspace of two apertures. Consequently, Eq.(5.5) is altered to be:

$$h = \frac{{d}_{i}^{2} * {h}_{i}}{π * s * ( {d}_{i} + s )} \qquad ( 7.9 )$$

The diameter and the width of the bulge were again measured with the help of an SEM image. As mentioned earlier the ETFE foil had a thickness of about 25 μm. Applying Eq.(7.9) using a diameter of 84.6 μm and a bulge width of 34.1 μm, the bulge height could be estimated to be 14.1 μm. This theoretical value was again verified by a Dektak profilometer measurement, cf. FIG. 8. The measurement of the 120 μm spaced array with its apertures resulted in an average bulge height of 19.05 μm. The difference between the theoretical and the experimental value of about 26% is caused by the assumptions made during the calculation. Furthermore, it is likely that material from the four other apertures surrounding the interspace was added to the measured bulge. Another method to verify the results is by turning the structure 49 degrees and take an SEM picture. The bulge can be seen clearly. However, it cannot be directly measured because of the angle in which the aperture is displayed. The actual bulge height is calculated by:

h=h_{turned}*sin α

h is the height measured on the picture and α was the angle with which the structure was turned. The result was a height of 17 μm. In summary, it may be concluded that the mathematical model developed is only applicable for a rough estimation of the bulge height when looking at single apertures. If more than one aperture contributes to the bulge, like it is the case in dense arrays, this estimation became too imprecise. The elements of uncertainty are the assumptions made to simplify the calculation and the unknown number of apertures which contribute to form the bulge at the interspace. To get a more exact value of the bulge height it is necessary to make a profilometer measurement or calculate it with the help of tilted SEM micrographs. 10 FIG. 8 shows an extract from the profilometer measurement of a horizontal row in a 120 μm spacing array; the displayed values are rounded off; the full graph has 18 peaks with an average height of 19 μm.

### Example 3

### Off Vector Delay and Spacing Consideration

The basic structure for this test was a hexagonal array with 10 apertures in each row and 11 rows. The distance from center to center (spacing) was chosen to be 250, 200, 150, 140, 130 and 120 μm. The optimal parameters for arrays with these spacings and an OVD of 1 μs were determined (Table 6).

TABLE 6
Overview of the optimized parameters for the production of
apertures with an OVD of 1 μs with an intensity of 0.4 W
Optimized parameters for the Off Vector Delay vs. spacing test

| | | |
|---|---|---|
| spacing in μm | Apertures | spot lase duration |

| | | | |
|---|---|---|---|
| 250 | All | 5 | ms |
| 200 | All | 5 | ms |
| 150 | 1^{st} of each row | 5 | ms |
| | 2^{nd} of 1^{st} row | | |
| | Remaining | 4.8 | ms |
| 140 | 1^{st} of each row | 5 | ms |
| | 2^{nd} of first row | 4.8 | ms |
| | 3^{rd} of first row | 4.5 | ms |
| | Remaining | 4 | ms |
| 130 | 1^{st} of each row | 5 | ms |
| | 2^{nd} of 1^{st} row | 4.8 | ms |
| | 3^{rd} of 1^{st} row | 4.5 | ms |
| | Remaining | 4 | ms |
| 120 | 1^{st} of 1^{st} row | 5 | ms |
| | 1^{st} hole of each row | 4.8 | ms |
| | 2^{nd} of 1^{st} row | | |
| | 3^{rd} of 1^{st} row | 4.5 | ms |
| | Remaining | 4 | ms |

### Example 4

### Increased Aperture Diameter

The purpose of these partitions was to investigate the bilayer formation for which purpose the density of apertures over the area was not of importance. Hence, 5×5 and 8×8 arrays were designed. The settings regarding intensity and spot lase duration were established. By initially having optimized the process to production of apertures being as small as possible, the production of larger apertures was straightforward. For this purpose the 200 mm lens with a focal spot diameter of 290 μm was more suitable. Two different aperture diameters were supposed to be produced: Apertures around 400 μm which were arranged in a 5×5 array with a center to center distance of 600 μm, and apertures with a diameter of 300 μm which were positioned in an 8×8 array with a spacing of 400 μm. Compared to the production of apertures with a diameter of 90 μm and below, here a higher power as well as a higher spot lase duration was needed. In the end, following parameters were found to fulfill the diameter requirements (Table 7).

TABLE 7
Production parameters for arrays of
apertures with increased diameter

| Diameter | Power | spot lase duration |
|---|---|---|
| 400 μm | 9% of cavity2 = 1.8 W | 12 ms |
| 300 μm | 6% of cavity2 = 1.2 W | 8 ms |

When fabricating these larger apertures care has to be taken to choose the right process direction. The effect of elliptical apertures mentioned above emerged. However, although structuring with the machine direction, the 400 μm apertures still appeared to be slightly oval. Single scaffolds of the resulting samples were taken to be examined. Therefore, SEM micrographs were taken and the diameter was calculated to prove that the desired diameter of the apertures could be achieved. However, the largest apertures were slightly oval. This deformed shape is suspected to be the result of the previously parallel aligned polymer chains and the higher energy input compared to the small apertures. Another factor could also be the OVD. Since the spacing was increased to 600 μm but the OVD was still at 1 μs, an influence of this parameter could not be excluded.

### Example 5

During the process of optimizing the production parameters for every material, optical inspection and geometrical measurements were performed to follow the progress. The results were used to decide, whether a further optimization was useful or if the material was not suited and had to be discarded. A parameter here was the achievable diameter which could be from 400 μm down to below 100 μm. It was measured using SEM pictures of the aperture hole and the software IMAQ Vision Builder 6.1 (National Instruments). Here, a line could be drawn through the hole and its length was given as the number of pixels. By measuring the scale bar in the SEM picture and relate the resulting number of pixels with the one measured in the hole, the diameter of the aperture could be calculated. This was done for the best results achieved with every material, i.e. PTFE, ETFE, FEP. With most materials the minimization of the aperture diameter stopped around the minimal focal spot diameter of 116 μm as given by the lens manufacturer and confirmed in the literature (Jensen et al. 2003 above). However, ETFE was an exception because minimal diameters of 100 μm or below could be made. With the help of the theoretical considerations made earlier above it was possible to estimate the height of the bulges surrounding the aperture. A SEM picture of an aperture in ETFE was used to measure both, bulge width and diameter of the actual hole. By using Eq.(5.5) the bulge height could be calculated. The production process was carried out using an OVD of 1000 μs. The intensity and spot lase duration had to be altered within the array. This resulted in having 4.2 W combined with 4 ms for the last row, 4.4 W and 5 ms for the first row and 4.4 W and 4 ms for the rows 2 to 7. By having an average aperture diameter of 303 μm, a bulge width of 65 μm and a thickness of the foil of 0.002 inch, the resulting bulge height should be 31 μm. This result was then verified by a measurement with a DekTak profilometer. The differences between the theoretical result and the measured value can be explained by the assumptions which were made to simplify the calculation. The actual bulges are neither completely elliptical nor evenly distributed. They are higher at the edge and gently flatten out on the outside. Furthermore, due to the nature of ablation, material is evaporated and thus not the entire volume of the hole is deposited as a bulge. In addition it is believed that the volume of the melted, ejected, and deposited polymer increases. This is caused by a lowering of the material's density during the heating process where polymer chains are cut into shorter chains without being ablated (SNAKENBORG, D., H. -KLANK, and J. P. -KUTTER. -Microstructure Fabrication with a CO₂/Laser System. -Journal of Micro-mechanics and Microengineering. (−2), pp-182). However, the measurement shows that the theoretical model is applicable to roughly estimate bulge heights.

### Example 6

### Use of CO₂ Laser Ablation in the Preparation of a Membrane Scaffold from an ETFE Film

General introduction to operation and laser parameters: The CO₂ Laser and therefore the fabrication of arrays, is controlled by the software package Win-Mark Version 4.6.2.5245 (SYNRAD Inc. Mulkiteo, Wash., USA). This software is provided by the manufacturer of the laser. The most important settings for fabricating described structures are the intensity of the laser beam, the Off Vector Delay (OVD) and the Spot Lase Duration (SLD). The intensity (or also referred to as power) controls how much of the overall power is used for the production. It can be chosen between 0 and 100% in steps of 0.1%. The specified output power of 50 W given by the machine supplier equals a value of 70 to 80%. The Off Vector Delay (OVD) sets the time when the laser is switched off between two production steps. Thus it gives the mirrors time to get settled over the starting point of a new structure before giving the command to “fire”. The software allows the OVD to be set to values between 0 and 80,000 μs. The last parameter of importance is the Spot Lase Duration (SLD). It defines the time for how long the laser stays on one spot. It can be chosen in ms and the maximum value equals 1 s. The following working example states the used values of all three of these important parameters: The used material was the Tefzel® 200LZ (DuPont®), an ETFE foil with a thickness of 0.002 inch (50.8 μm). The structure which was fabricated consisted of 64 apertures which were arranged in a rectangular array of 8 columns times 8 rows. The average diameter of the apertures was estimated to be 300 μm+/−5 μm and the center to center distance of the apertures (also referred to as spacing) was chosen to be 400 μm. The task of having equally sized apertures over the whole structure made it necessary to change the laser parameters within the array. This can be explained by the thermal energy which is introduced by the production of neighbouring apertures—this means that apertures in the middle and at the end of the array can be produced with less energy than at the beginning. This relationship between the apertures influences all three main laser settings. The OVD was chosen to be 1000 μs instead of 1 μs like it is used with the thinner Tefzel 100LZ foils. The purpose was to give the material time to cool down before “shooting” the next aperture to avoid deformation of the apertures. The SLD was chosen to be 5 ms for the first horizontal row and 4 ms for all subsequent rows. The power was altered from 22% (4.4 W) to 21% (4.2 W) for the last row. These settings (Table 5) made it possible to produce an array with the preferred characteristics including a smooth rim with a bulge of about 17 μm (see also FIG. 8):

TABLE 8
Summary of the used settings for the preparation of
a membrane scaffold with 8 × 8 apertures of 300
μm ± 5 μm diameters and 400 μm spacing
in a rectangular array in ETFE foil with a thickness of 0.002 inch

| Location | Intensity | SLD | OVD |
|---|---|---|---|
| first row horizontal | 22% | 5 ms | 1000 μs |
| last row horizontal | 21% | 4 ms | |
| all others | 22% | 4 ms | |

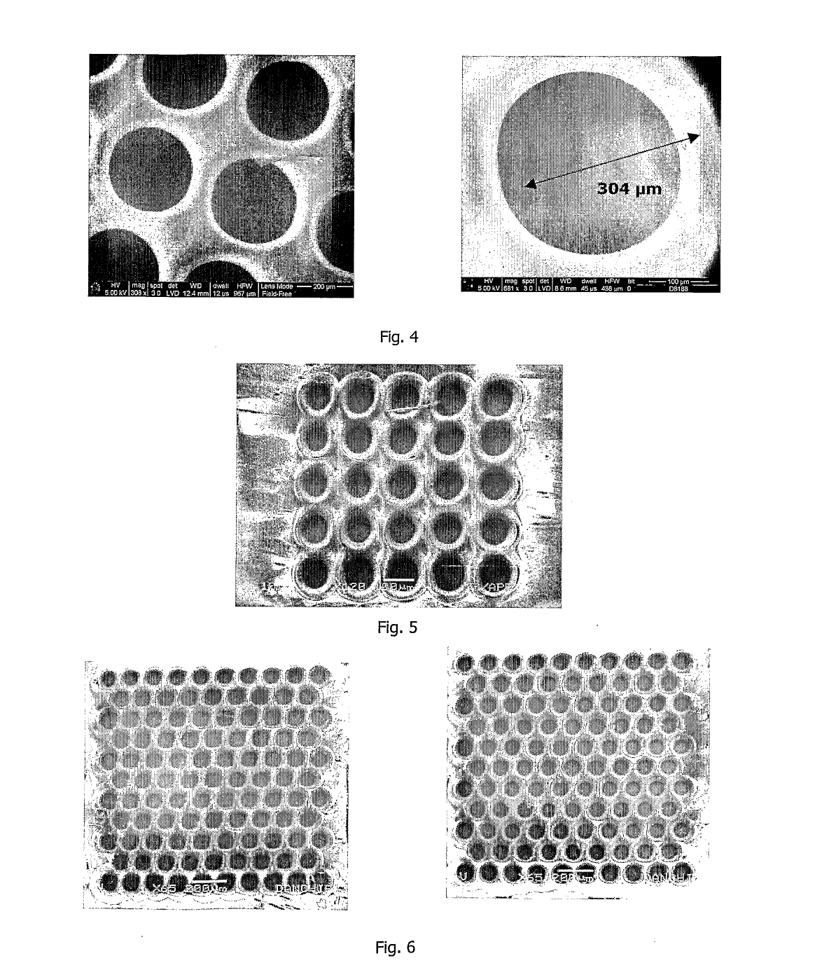

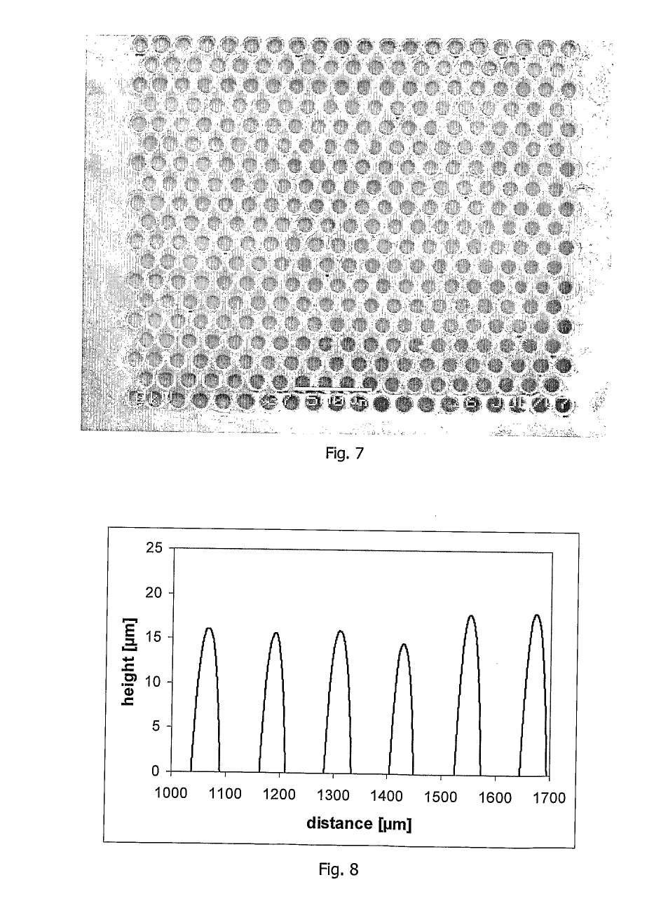

The same laser settings, except using an OVD of 1 μs, were used in the fabrication of membrane scaffolds having the same array configuration but prepared in Tefzel® 100LZ (DuPont®) foil having a thickness of 0.001 inch (25.4 μm). Laser settings will ideally have to be optimized for the preparation of membrane scaffolds having other specifications. Typically, OVD settings are increased with increasing thickness of the ETFE film to avoid deformation of the apertures. Due to higher power and SLD settings when structuring thicker films more thermal energy is absorbed by the material and thus a longer cooling time between the productions of two neighbouring apertures is preferred. FIG. 4 is a SEM picture of the central aperture array (average diameter about 300 μm) of a scaffold of the invention, where the left side is a section of a 8×8 array; right side is a single aperture of such an array. The pictures were taken using a FEI Nova 600 NanoSEM. The use of a low vacuum made it possible to take clear pictures of the non-conducting polymer by scanning electron microscopy (SEM). An array of near circular apertures having perfectly smooth rims is shown. The lighter shades of the rims indicate bulging to an extent of about 10 to 40 μm above original foil. The actual bulges are a bit wider than the visible lighter shades. These lighter shades show the inside (the raising) part of the bulge. As can be seen on the right picture the lighter shade stops almost at the top and the other side of the bulge is darker again.

Conclusion:

The CO₂ laser enables preparation of the membrane scaffolds of the invention having apertures with diameters in the range from about slightly less than 80 μm to about 400 μm and above, the desired rim smoothness and rim bulging, and it also enables very close spacing of the apertures, i.e. producing an aperture area of up to 44% using the rectangular arrangement of the apertures and 47% using a hexagonal arrangement relative to the entire functional area of the scaffold. Moreover, the laser enables fast production of smaller samples, e.g. the production of a scaffold having an 8×8 aperture array is done in less than 3 seconds.

### Example 7

### Method and Device for the Preparation of Auto Painted Membrane

Preparation of a composite bio-mimetic membrane using a circular disk (diameter 29 mm) scaffold of the invention having a rectangular 8×8 aperture array (each aperture has an average diameter of 300 μm) with a centre to centre distance of 400 μm formed in ETFE film (Ethylene-TetraFlouroEthylene, 100LZ ETFE of 0.001 inch (25.4 μm) film thickness, DuPont®) using the CO₂ laser ablation according to the procedures described in Example 6.

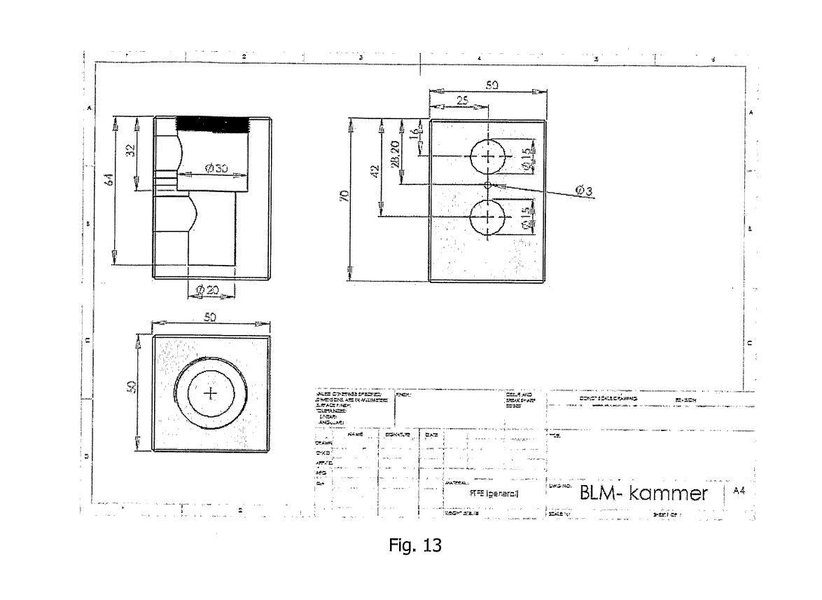

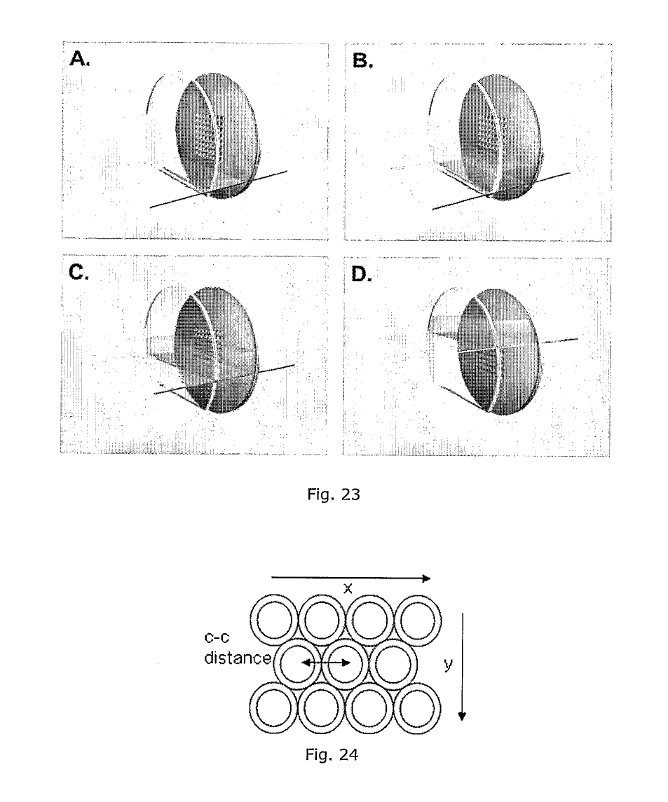

The APM principle: The APM principle is sketched in FIG. 9 and in FIG. 23 where the basics of the Auto-Painted Membrane (APM) technique is shown with one or 64 apertures respectively. FIG. 9 shows a sectioned schematic side view through the middle of an assembled two-cell Teflon chamber (the APM-1 chamber, cf. FIG. 13). In steps 1-3 the buffer level in the cis chamber (left-hand chamber in FIG. 9) is raised above the aperture, thus creating a lipid bilayer (red line, step 3) by the parallel raising of the DPhPC/decane layer (red square, step 1-3). It has been found that prepainting the aperture array of the scaffold with a solution of the amphiphilic substance (block co-polymer or lipid) in a suitable solvent, such as decane, hexane, etc., before mounting in the APM chamber facilitates the formation of membranes in the apertures.

Cleaning APM-1 chamber parts: The Teflon parts of the APM-1 chamber were cleaned with 3 successive washes in 96% ethanol, Folch mixture and chloroform, followed by a thorough rinse in Millipore water. Viton A (flourodipolymer, DuPont) seals were cleaned once in 50% (v/v) ethanol for 10 minutes in an ultrasonic bath (BRANSON 1510, Buch&Holm) followed by a 10 minute ultrasonic rinse in Millipore water. Scaffolds were washed 3 times successively in 60% (v/v) ethanol, hexane, and water.

Pre-painting ETFE scaffolds: The pre-painting solution used in this study consisted of 1,2-Diphytanoyl-sn-Glycero-3-Phosphocholine (DPhPC, Avanti Polar Lipids, Inc., Alabaster, Ala.) (50 mg/ml) dissolved in n-decane (Sigma®). The same lipid solution was used as bilayer forming solution (BFS). Cleaned and dried ETFE scaffolds were first pre-painted once on both sides by adding and distributing a small droplet of pre-painting solution and then leaving the scaffold to dry under a fume hood. The following 4 pre-paints involved maintaining open apertures with a stream of N2: By carefully blowing on the apertures of the scaffold with a stream of N2 after addition of the pre-paint droplet the solvent could be evaporated from the pre-painting solution, while keeping the apertures open.

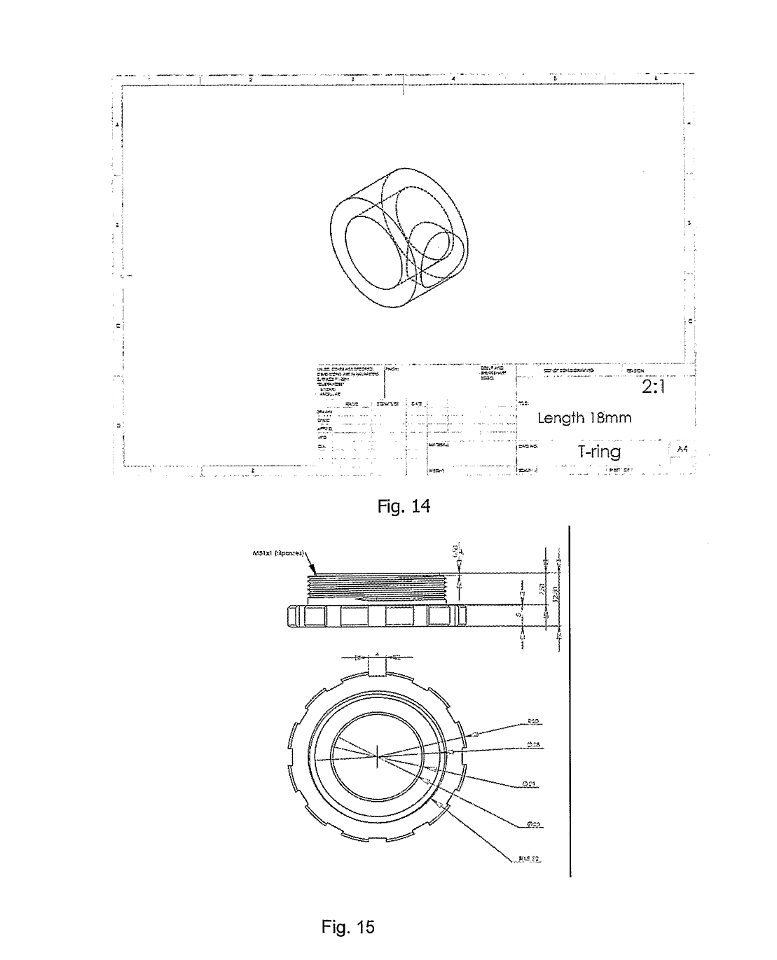

Assembling APM-1 chamber parts: The assembly of the individual inner elements of the APM-1-chamber is shown below (FIGS. 12, 12a). The inner elements all have approximately a 29.9 mm outer diameter to fit snugly into the cylindrical 30 mm diameter tube of the cis chamber (left-hand chamber in FIG. 12a), and rest upon the ledge created by the interface of the cis chamber and the cylindrical 20 mm diameter tube of the trans chamber. A 5 mm thick Teflon cylindrical tube (arrow 8 in FIG. 12, see also FIG. 14) provides the link to the annular brass screw (perimeter thickness 7 mm) generating sufficient pressure from the exterior of the chamber to obtain water tight sealing. Thus both the cis and trans chambers have identical inner diameters of 20 mm when the APM chamber is assembled.

A circular, 2 mm thick Teflon spacer (arrow 5 in FIG. 12) with a 2 mm slit is positioned with the opening at the top to allow for entering membrane forming solution behind the cut glass (arrow 7 in FIG. 12) with a Hamilton inserted through a 3 mm cylindrical opening through the top part of the APM chamber (FIG. 13).

The inner diameter of the inner Viton seals (arrows 2 in FIG. 12) and Teflon spacers (arrows 1 and 5 in FIG. 12) as shown here is about 8 mm to better stabilize the scaffold (arrow 4 in FIG. 12) when made of the thin 100LZ film. The scaffold support material (arrow 3 in FIG. 12) is comprised of a about 250 μm thick sheet of regenerated cellulose sheet having a molar mass cut off of 10 kDa and a contact angle of 10.3 °, DSS-RC70PP, Alfa Laval, Denmark.

When sealed the chamber space between the cover slip (arrow 9 in FIG. 12) and the scaffold (arrow 4 in FIG. 12) constitutes the cis chamber. When using scaffolds made of thicker film material, e.g. about 50 μm (0.002 inches), the parts 1, 2, and 5 can be of the same inner diameter as the other parts. Optimal sealing is achieved by applying a thin layer of silicone grease (High Vacuum Grease, Dow Corning) to the inner Viton seals (arrows 2 in FIG. 12) prior to assembly.

The annular brass screw having an inner diameter of 20 mm (not shown, cf. FIG. 15) secures tight sealing from the right end as shown by the arrow. It is possible to visually follow the lowering and raising of buffer levels in the cis chamber through the opening in the annular sealing screw.

### Example 8

### Incorporation of Functional Valinomycin into Auto-Painted Membranes (APM's)

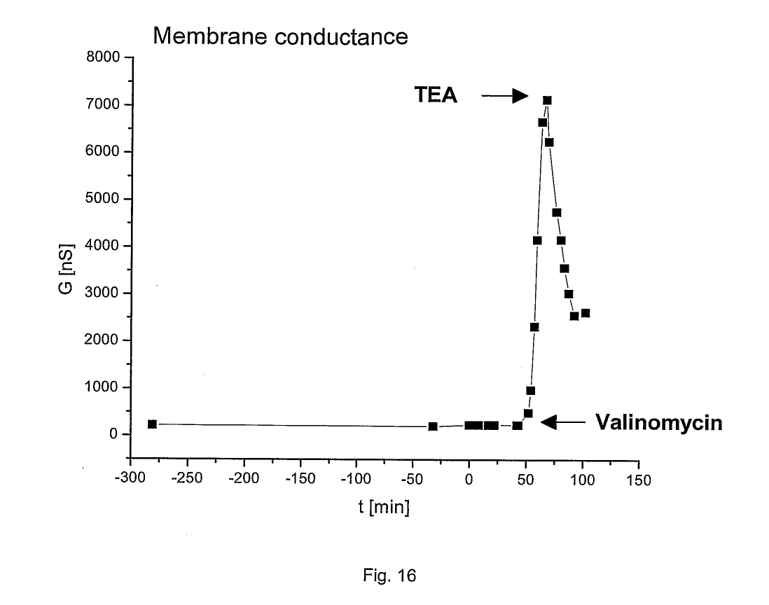

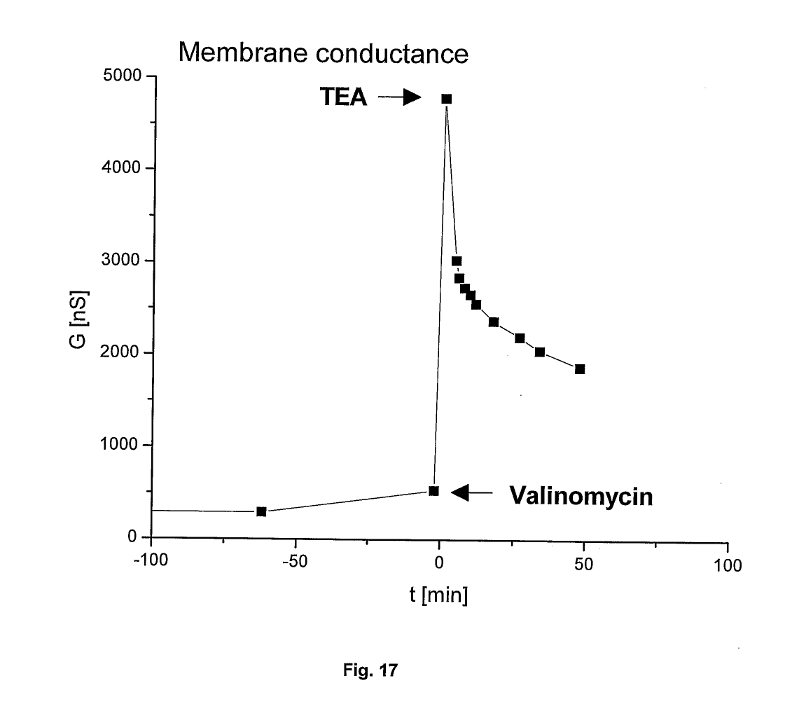

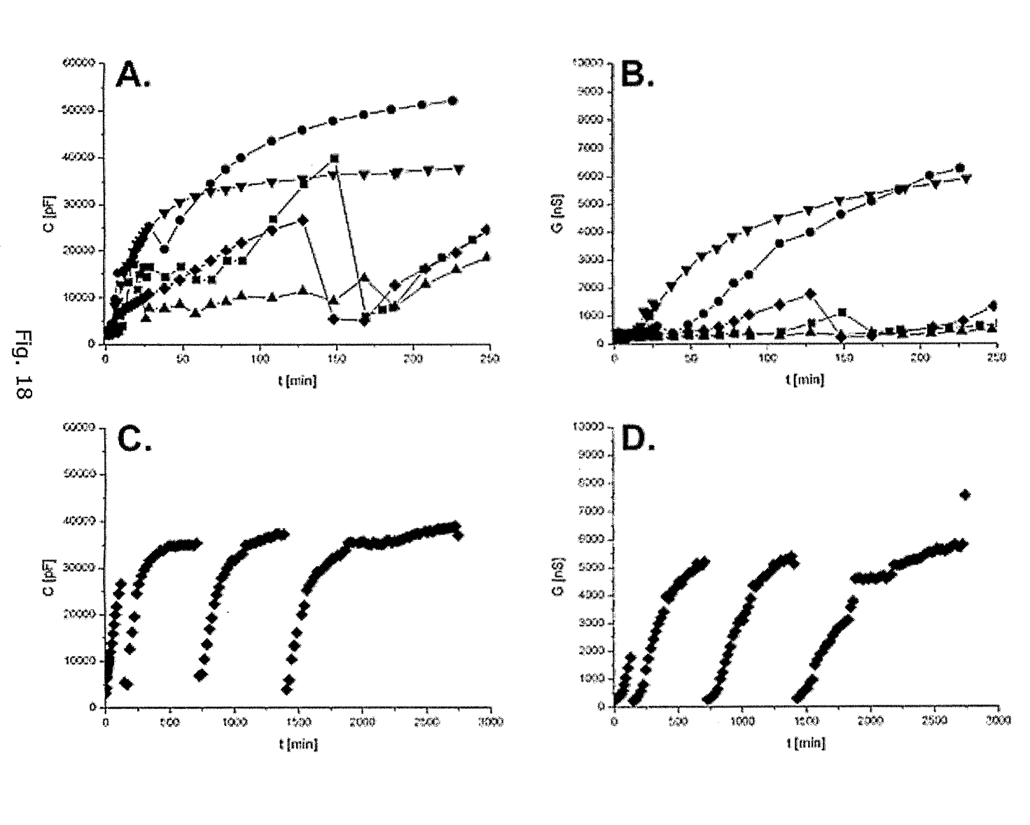

In a 8×8 ETFE membrane array having aperture diameters of about 300 μm In this study we incorporated the potassium ionophore valinomycin (Mary Pinkerton, L. K. Steinrauf and Phillip Dawkins: “The molecular structure and some transport properties of valinomycin”. Biochemical and Biophysical Research Communications VOL 35 Issue 4 512-518) in very stable (>100 hours) DPhPC membrane systems (lipid bilayers) formed in a membrane scaffold of the present invention, and subsequently we reversed the valinomycin induced increase in conductivity by adding tetraethylammonium chloride (TEA), a known inhibitor of potassium ionophores (Robert J. French, Jay B. Wells: “Sodium Ions as Blocking Agents and Charge Carriers in the Potassium Channel of the Squid Giant Axon”. BIOPHYS. J. VOL 54 1053-1063). Voltage clamp measurements were performed in an in-house manufactured Faraday cage. The primary electrical setup consisted of a headstage (HS-2A, Eastern Scientific LLC) and an amplifier (PICOAMP-300, Eastern Scientific LLC). Data acquisition was done with a combined oscilloscope/analog-digital converter (ADC-212, Pico technology. A 200 mM KCl solution served as electrolyte. Valinomycin powder (Sigma) was dissolved in 96% ethanol to yield a 2 mg/ml (1.8 mM) working solution (WS), which was stored at 4° C. A 16 mM TEA working solution was prepared in 200 mM KCl and stored at 4° C. 2-10 μl Valinomycin WS was added to the small APM-1 chamber volume through the slit in Teflon spacer (arrow 5 in FIG. 12) between the ETFE scaffold and the first glass coverslip (≈0.5 mL), cf. FIG. 12, in APM-1 setups where the APM's displayed constant membrane characteristics over several days. To reverse the valinomycin-induced conductance, TEA working solution was added to the small chamber volume in molar excess. Results: The graph in FIG. 16 shows reversing valinomycin induced increase in conductance by adding TEA. Experiments performed on 13 day old membrane. 10 μL 1.8 mM valinomycin WS added to the small chamber volume (0.5 mL) at t=52 min corresponding to ˜32 μM final valinomycin concentration. 500 μL 16 mM TEA WS added to the small chamber volume between t=69 min and t=2 min corresponding to ˜8 mM final TEA concentration. The graph FIG. 17 shows the reversal of valinomycin induced increase in membrane conductance by adding TEA in molar excess. Experiments were performed on a 4 day old composite membrane. 10 μL 1.8 mM valinomycin was added to the small chamber volume (0.5 mL) at t=0 min corresponding to ˜32 μM final valinomycin concentration. 200 μL 16 mM TEA WS added to the small chamber volume at t=5 min corresponding to ˜4.5 mM final TEA concentration.

Conclusion: In this study we have demonstrated facilitation of incorporation of valinomycin into Auto-Painted Membranes created in ETFE 8×8 300 μm diameter aperture diameter arrays, and subsequently reversal of the Valinomycin induced increase in conductance by adding TEA. Control experiments have shown that addition of and TEA alone does not significantly affect membrane characteristics (not shown). Our results in this study confirm that we can manipulate membranes in ETFE arrays created by use of the APM technique.

Upon reversing the valinomycin induced increase in membrane conductance by adding TEA, we observe again a significant increase in membrane conductance, which is followed by membrane failure (not shown). It is believed that this increase in membrane conductance followed by membrane failure to be a result of an excessive amount of valinomycin in an unstirred layer close to the membrane.

### Example 9

### A Diagnostic Kit for Detection of Serum CD 95/Fas Ligand

A composite biomimetic membrane will be prepared in the APM-1 chamber as described in Example 8. A fluorescently labeled, e.g. with an environmentally sensitive probe, such as BadanR or LaurdanR, CD-95 receptor (Fas protein, 5 catalogue No. 198749, ICN Biochemicals & Reagents 2002-2003) will be prepared in an emulsion according to Beddow et al. (Anal. Chem. 2004, 76, 2261-2265) and added to the membrane through the slit in Teflon spacer (5) for direct reconstitution in the membrane. A serum sample extract containing Fas ligand to be tested will be added to the membrane. Following binding of the Fas ligand extract to the prepared membrane the membrane will be transferred to either a microscope or a spectrophotometric plate reader (Wallac Victor2) for examination. Quantification of binding will be based on an internal standard of known fluorescence.

### Example 10

### Preparation of Membranes with Functional Valinomycin

A Synrad Duo 48-5S Duo Lase carbon dioxide laser with a specified power output of 50 W (Mulkiteo, Wash., USA) and equipped with a 200 mm 20 focal length lens was used to fabricate partitions with 8×8 rectangular arrayed apertures in ETFE LZ200 film (50.8 μm thickness). The average diameter was 301±5 μm (n=5) positioned in the array with an aperture centre-to-centre spacing of 400 μm. The 8×8 array was placed in the middle of a circle with a diameter of 29 mm. The apertures were produced with an intensity of 1.2 W and a spot lase time (impact time of the beam) of 8 ms. The ETFE film was placed in a custom produced sample holder made of polymethyl methacrylate. A clearance was situated in the middle of this fixture where the laser beam hit the sample. Thereby, it was assured that no underlying material interfered with the production process.

A scanning electron microscope (SEM) (Jeol JSM 5500 LV SEM from GN nettest) was used for imaging. It is capable of a lateral resolution of 30-50 nm and a magnification up to ×300,000. The acceleration voltage can be set between 1 to 30 kV. The SEM has a reproducibility and accuracy in lateral distance measurements better than 5.0%. SEM images of the produced CO₂ laser percussion drilled EFTE partitions showed that the apertures were symmetrically positioned in the 8×8 array with slight elliptical apertures having nicely rounded edges (FIG. 22).

The lipid bilayer chamber design is depicted in FIGS. 12 to 15. The complete chamber setup consists of a main Teflon chamber with two asymmetrical drilled holes having diameters of 20 and 30 mm respectively, a 30 mm diameter cylindrical Teflon tube (5 mm thickness), two 30 mm circular Teflon inter spacers where one has a 2 mm slit, six Viton O-ring seals, two coverslip glasses where one is cut, and a brass screw to tighten the bilayer chamber. The inner elements consisting of a porous cellulose support, ETFE partition, Teflon spacers, circular glass cover slips and Viton O-rings fit into the cylindrical 30 mm diameter tube of the cis chamber, and rest upon the ledge created by the interface of the trans chamber and the cylindrical 20 mm diameter tube of the cis chamber. The 5 mm thick cylindrical Teflon tube provides the link to the 20 annular brass screw (perimeter thickness 7 mm) generating sufficient pressure from the exterior of the chamber to obtain a water tight sealing. Thus the cis and trans chambers have identical volumes. A circular, 2 mm thick circular Teflon spacer with a 2 mm slit is positioned with the opening at the top of the chamber that allows for entering bilayer forming solutions into the lipid bilayer chamber with a Hamilton syringe.

Pre-Painting of Scaffold using the Airbrush Technique

The lipid solution for pre-treatment of ETFE LZ200 partitions (pre-painting) and for the bilayer forming solution consisted of 50 mg/ml of 1,2-Diphytanoyl-sn-Glycero-3-Phosphocholine (DPhPC) in decane. 30 DPhPC (2 ml) in chloroform (10 mg/ml stock) was evaporated under nitrogen gas and the dry lipid was resuspended in 400 mL decane. The bilayer forming solutions were stored at −20° C. until use. For fluorescent microscopy the lipid solutions were added 1 mol% of 1-Oleoyl-2-[6-[(7-nitro-2-1,3-benzoxadiazol-4-yl)amino]hexanoyl]-sn-Glycero-3-Phosphocholine (NBD-PC). Pre-painting of ETFE partitions was carried out by the addition of approximately 5 mL of DPhPC in decane (50 mg/ml) using a glass Pasteur pipette to both sides of the ETFE partition. The ETFE partitions were left to dry for 10 min followed by applying a gentle stream of nitrogen gas to both sides the partition to ensure opened apertures. The prepainting step was repeated five times, and the pretreated ETFE partitions were stored in a vacuum desiccator until use. Another pre-painting strategy was developed to provide a more controlled and uniform deposition of the pre-painting solution (50 mg/ml DPhPC in decane) to the ETFE partition aperture arrays, and was based on airbrushing the pre-painting solution onto the ETFE partition sides. The airbrush setup consisted of an airbrush (type: MAS G41, TCPGlobal) connected to a nitrogen gas flask and mounted onto an aluminum track with a ruler. The airbrush was positioned with a distance of 45 mm from the airbrush nozzle to the ETFE partition. The partition was mounted on a brass housing that was connected to a low capacity vacuum pump. Partitions were placed on the brass housing and the vacuum pump turned on briefly to fix the partition in position during the pre-painting procedure. The 0.6 ml gravity feed cup of the airbrush was filled with pre-painting solution (100 μl) and the pre-painting solution was deposited onto the ETFE partitions as a fine mist using a nitrogen pressure of 15 psi. The partitions were applied pre-painting solution on each side consequitive times with an interval of 30 s to give a thin uniform coverage of prepainting solution on the ETFE partitions.

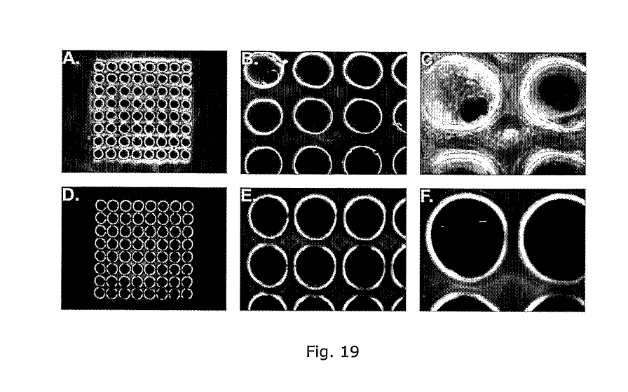

Fluorescent imaging was performed on a Zeiss Axiovert 200M epifluorescence microscope (Carl Zeiss, Jena, Germany) equipped with a monochrome Deltapix DP450 CDD camera (Deltapix, Maalov, Denmark). Imaged were acquired using Deltapix DpxView Pro acquisition software (Deltapix, Maalov, Denmark). Objectives used were air corrected Plan-Neofluar 2.5×/0.075 Numerical Aperture (NA), 10×/0.25 NA and 20×/0.40 NA respectively. FIG. 19 shows 6 fluorescent images of traditional and airbrush pretreated multiple apertures. Epifluorescence images of the ETFE 8×8 apertures array partitions pre-treated with pre-painting solution (50 mg/ml DPhPC in decane) with 1 mol % of the fluorescent lipid NBD-PC with two different methods. Images A), B), and C) show the pre-treatment of the ETFE partition by the traditional pre-painting method using a glass Pasteur pipette for 5 consecutive times on both sides. Objectives used were A) 2.5×, B) 10× and C) 20×. Images D), E), and F) show the pre-treatment of ETFE partitions by the air-brushing the pre-painting solution on both sides for 20 consecutive times with 30 s intervals. Objectives used were 15 D) 2.5×, E) 10×, and F) 20×.

The lipid bilayer chamber was assembled with the ETFE partition prepainted using the traditional method and a circular regenerated cellulose sheet (DSS-RC70PP, Alfa Laval) with diameters of 29 mm. The regenerated cellulose was included in the multiple bilayer formation technique to provide a porous support structure for BLM formation. This semisupported bilayer formation strategy was chosen to minimize the hydrostatic pressure between the trans and cis chamber upon establishment of lipid bilayers. Once assembled, the ETFE partition was by design located at the center of the circular interface between the cis and trans lipid bilayer chamber. The trans and cis chambers were filled with 7.5 ml of a 200 mM KCl, pH 7.0 solution, and the lipid bilayer chamber was then placed in a Faraday cage and the silver/silver chloride electrodes placed in the electrode wells. The level in the cis chamber was lowered to the beginning of the cut glass coverslip by aspiration of approximately 7 ml aqueous KCl solution using a plastic Pasteur pipette (FIG. 23A). A Hamilton pipette was filled with 100 mL of DPhPC in decane (50 mg/ml), and the bilayer forming solution was applied to the space between the cut cover slip glass and the combined partition and regenerated cellulose support of the cis chamber through the 2 mm slit in circular Teflon spacer of the assembled chamber. The level of the aqueous solution in the cis chamber was slowly raised by adding approximately 7 ml aqueous KCl solution using a plastic Pasteur pipette (FIG. 23B-C). During the raising of the aqueous KCl solution the stepwise formation of BLMs in the partition aperture arrays was recorded by measurements of the capacitance and conductance signals. The primary electrical setup consisted of a Model 2400 Patch Clamp Amplifier with a headstage containing 10G/10M feedback resistors (A-M 15 Systems, Inc., Wash., USA) and a Thurlby Thandar Instruments model TG2000 20 MHz DDS function generator (RS components Ltd, Northants, UK).

Data acquisition was done with a combined oscilloscope/analogdigital converter (ADC-212/50, Pico technology) connected to a laptop computer. Sampling frequency was 50 Hz and the low pass filter corner frequency of 1 kHz. Capacitance and conductance measurements were performed by applying triangular and rectangular voltage clamp waveforms. Reference measurements of the combined regenerated cellulose and ETFE partition were made prior to formation of the multiple BLMs. Formation of planar lipid bilayers across multiple aperture ETFE partitions was performed by the method outlined in FIG. 23. This method proved to be reliable in the sense that multiple BLMs (64 apertures) were generally established by a single or two lowering and raisings (FIG. 23), and with a success rate above 95%. For all multiple aperture bilayer experiments initial conductance values were in the range of 250-900 nS and capacitance values in the range of 2000-6000 pF. We evaluated the capacitance and conductance contributions of regenerated cellulose, and of ETFE film without apertures and regenerated cellulose mounted in the bilayer chamber respectively. The regenerated cellulose had a conductance of 9165.9±23.0 nS and no measurable capacitance (n=5), whereas the ETFE film plus cellulose had a conductance of 127.0±1.6 nS and a capacitance of 2336.4±19.0 pF (n=5). Given that the capacitance and conductance values for the multiple bilayer experiments are comparable to the reference values of the overall system, the initial measured capacitance and conductance values in the multiple aperture bilayer experiments are interpreted as being from the system alone and is due to the effective sealing of the apertures by the bilayer forming solution. The observed fluctuations in the initial conductance and capacitance values were due to variations in chamber assembly (e.g. tightening of the brass screw, silicone grease deposition, etc). Thus, the initial capacitance and conductance values and the initial value fluctuations observed for formation of multi array lipid bilayers reflect the inherent capacitance and conductance properties of the bilayer chamber and assembly. Following lipid membrane sealing across the apertures, lipid bilayers start to form and expand inside the apertures. The thinning of lipid membranes into bilayers give rise to an increase in the observed capacitance (FIG. 18A), while an increase in conductance is observed when the sealing properties of the BLMs start to fail (FIG. 18B). Interestingly, some membranes (“10%) exhibited a capacitative discharge and recharge cycling behaviour with a time course of approximately 500 min, which is also reflected in the time course of the conductance. This behaviour of BLMs formed across multiple aperture partitions was observed as a repetitive cycling in capacitance and conductance values from the initial lipid bilayer formation values of around 2000-5000 pF and 250-900 nS, and to lipid bilayer values of 26000-38000 pF and 2500-5800 nS respectively (FIGS. 18C and 18D). In general, lipid bilayers formed across multiple aperture ETFE partitions were stable for 200-300 min before breakdown, while some membranes (approx. 40%) lasted for 1-3 days when left with voltage potentials≧±100 mV. However, the membrane thinning curves varied considerably between experiments with average capacitance and conductance values of 31607.6±13425.7 pF and 2947.0±2898.8 nS (n=5) at 250 min (FIGS. 18A and 18B).

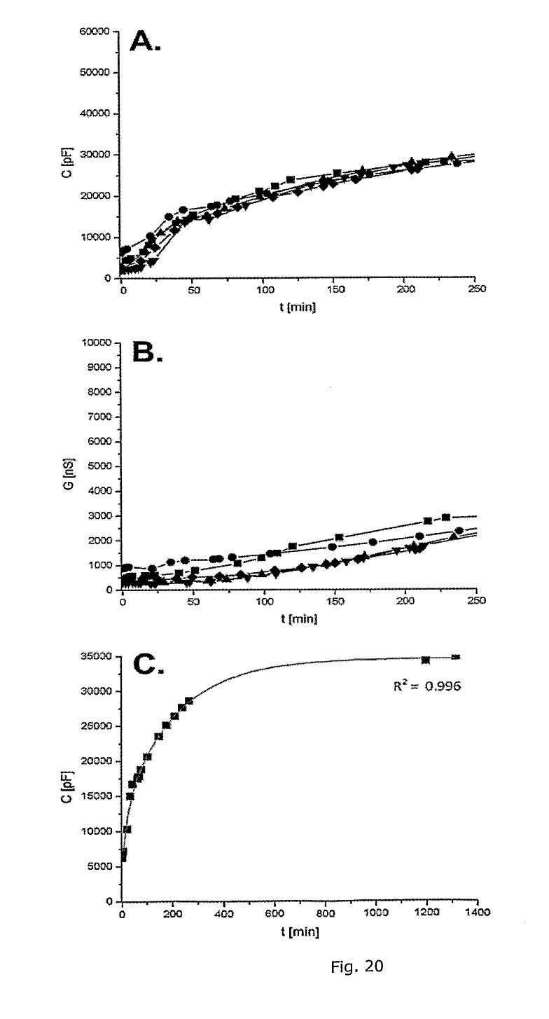

A reason for the low experimental reproducibility of BLM characteristics from establishment of the lipid membranes to lipid bilayers could be influenced significantly by variations in the amount and homogeneity of lipid depositions during the pre-treatment process. Therefore, fluorescent images were acquired of 8×8 array ETFE partitions following pre-painting with DPhPC in decane (50 mg/ml) with 1 mol % of the fluorescent lipid NBD-PC for five consecutive times on each side followed by opening of apertures under a gentle stream of nitrogen gas. Results showed that the pre-painting solution was deposited inhomogeneous on the partition surface, and several apertures were consistently partial or completely occluded by the prepainting solution in spite of the applied nitrogen to open the apertures (FIGS. 19A, 19B and 19C). Although the majority of apertures were open, they had a varied degree of lipid deposition around the rim of the apertures protruding into the aperture, thus reducing the effective aperture diameter. This could also be a factor negatively influencing the reproducibility (FIGS. 23B and 23C). To circumvent the inhomogeneous lipid pre-treatment depositions, an air-brushing pre-treatment technique was developed. The airbrush technique for pre-treatment of partitions, e.g. as described above, was able to deposit lipids onto the ETFE partition in a homogenous and controlled manner by using a nozzle to partition distance of 45 mm, a nitrogen pressure of 15 psi and painting intervals of 30 s. Fluorescent images of ETFE partitions pretreated for twenty consecutive times on each side showed that apertures were homogeneously covered by a fine thin layer of lipids (FIGS. 19D, 19E, and 19F). Apertures were coated with lipid without being occluded, and aperture rims exhibited a uniform coating without lipid depositions protruding into the apertures (FIGS. 19E, and 19F). Multiple aperture bilayer experiments revealed that enhanced reproducibility were achieved by establishing BLMs across the airbrush pre-treated multi aperture ETFE partitions (FIG. 20A). Following a short lag phase (˜2-10 min) the multiple formed lipid membranes thinned in a time dependent manner reaching maximum capacitance values of 28529.7±1421.7 pF at around 250 min (n=5) (FIG. 20A).

The conductance values were relatively stable (540.9±128.2 nS) during the time course of 100 min at which point the conductance increased 15 to 2323.3±460.9 nS during the time course from 100 min to 250 min. In the minutes prior to membrane rupture an abrupt increase in conductivity was commonly observed.

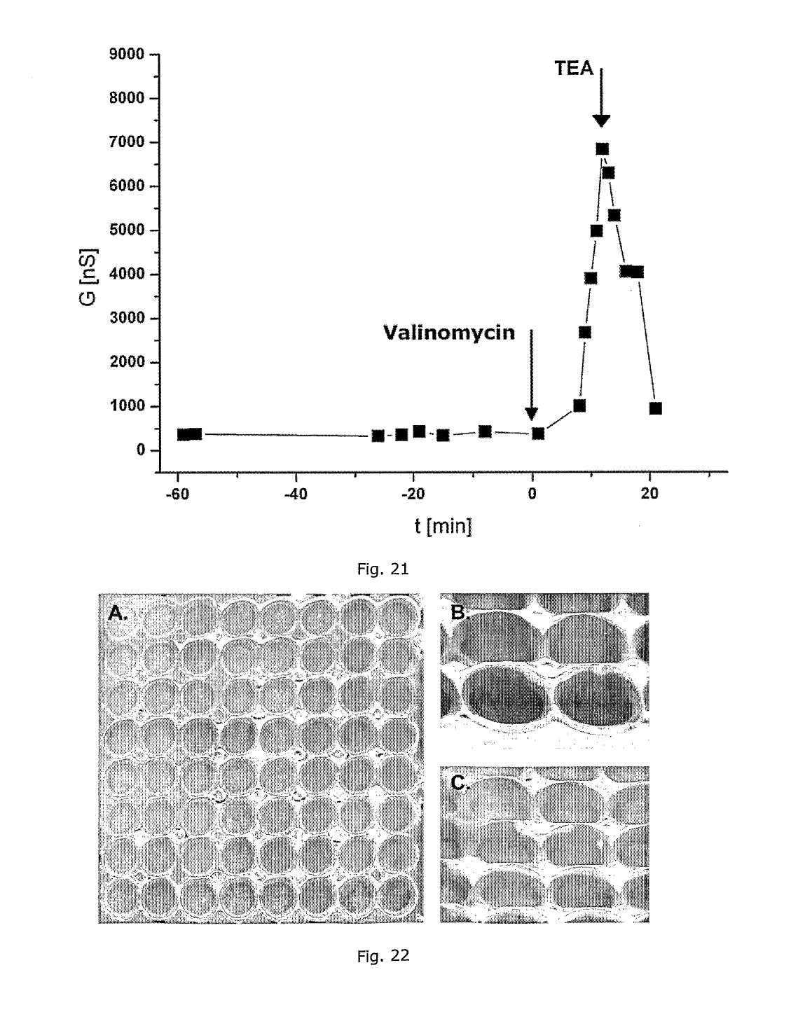

Valinomycin was dissolved in 96% ethanol to yield a 1.8 mM working solution, which was stored at 4° C. until use. Tetraethylammonium (TEA) working solution (16 mM) was prepared in 200 mM KCl and prepared immediately before use. Valinomycin (1.8 mM) was added (10 μl) to the small chamber volume between the ETFE partition and the first glass coverslip in the chamber setup (volume 0.5 ml), corresponding to a final valinomycin concentration of ˜32 μM. Valinomycin incorporation was only performed on multiple BLMs displaying constant membrane characteristics for more than 60 min. To reverse the valinomycin-induced conductance, TEA working solution was added (200 μl) to the small chamber volume, corresponding to ˜4.5 mM TEA. To ensure that bilayers are formed across the multi aperture partitions the potassium ion-selective cyclodepsipeptide valinomycin were added (32.0 μM final concentration) to lipid bilayers displaying a stable conductivity for more than 60 min. Following addition of valinomycin to the chamber an abrupt increase was immediately observed indicating functional reconstitution of valinomycin cyclodepsipeptides into the bilayers formed across the array of 8×8 aperture (300 μm diameters) partitions. The effect of valinomycin could effectively be reversed by the addition of 5 the channel blocker TEA (4.5 mM final concentration) (FIG. 21). In contrast, addition of ethanol or TEA alone to formed lipid bilayers to final chamber concentrations of up to 1% and 5.9 mM, respectively, did not significantly affect membrane characteristics (data not shown). Combined, these results strongly indicates that stable lipid bilayers are in fact formed across the multi array aperture partitions and that lipid bilayer spanning channels can be functionally inserted into the formed lipid bilayers. Giving the total aperture area of 0.045 cm² for 64 apertures with average diameters of 300 μm and a specific capacitance of 0.4-0.6 μF/cm² previously determined for solvent containing lipid bilayers, the capacitance for multiple formed bilayer lipid membranes were expected to be in the range of 18095 to 27143 pF. Therefore, the capacitance values of 29126.6±691.9 pF found in this study (FIG. 20A) indicate that the total lipid bilayer area is somewhat 6.4-37.6% larger than expected with a specific bilayer capacitance of 0.4-0.6 μF/cvm². This indicates that either the membranes formed by the technique presented herein is thinner compared to the conventional manually painted lipid membranes or a reservoir of lipid is present on the sides of the partition when bilayer lipid membranes are formed across the multi aperture ETFE partitions, or a combination of both.

### Example 11

### Testing of Different ETFE Films as Scaffold Material

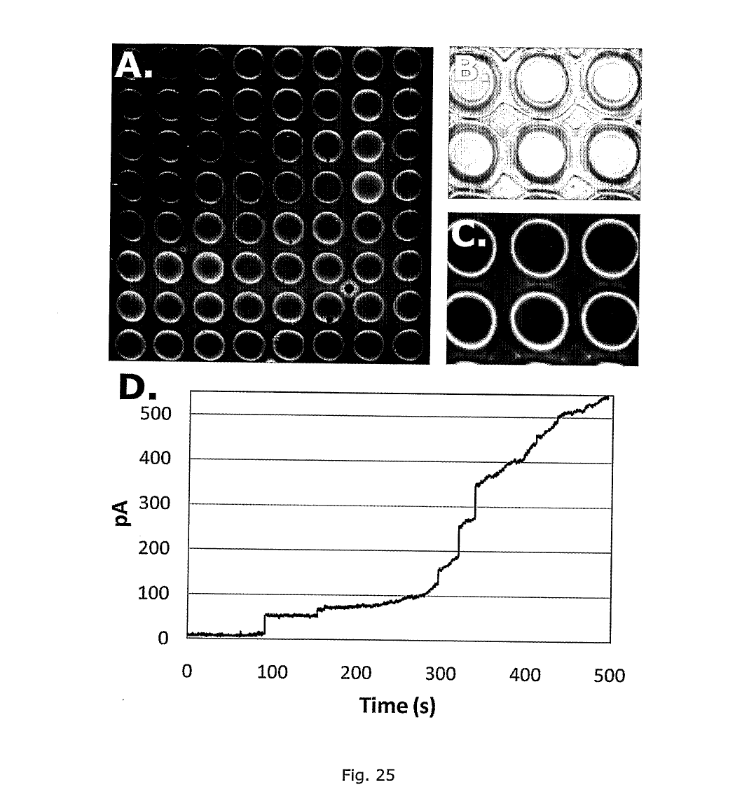

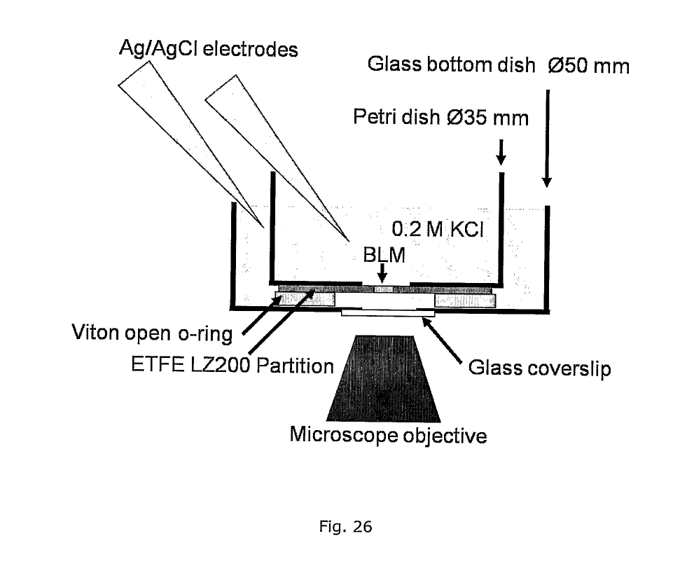

Scaffolds having 300 μm diameter apertures in a rectangular 8×8 arrays and a centre-to-centre distance of 400 μm were prepared in two different ETFE film materials: Fluon 50N and Tefzel LZ200 both having a 50.8 μm thickness. BLMs were prepared in these scaffolds to make composite biomimetic membranes using the lipid, solvent, and aqueous electrolyte solution materials described in Example 10 above in a horizontal chamber setup, cf. FIG. 26 without scaffold prepainting and where a hydrophobic fluorescent material, i.e. NBD-PC (a fluorescent lipid analogue) had been added to the lipid setup adapted for microscopic visualisation. Fluorescence images were obtained using a 2.5× objective and virtually identical BLMs were observed, cf. FIG. 25 wherein it can be seen that identical composite membrane arrays can be formed in different ETFE brands.

### Example 12

### Incorporation of Alpha-Hemolysin in Composite Biomimetic Membrane

Aim of experiment was to show that functional establishment of black lipid membranes (BLMs) can be performed by inserting membrane spanning peptides and proteins. A protein that inserts spontaneously into functional BLMs is α-hemolysin (αHL), which forms a heptameric protein complex when reconstituted into established membranes. Protein incorporation can be followed because the insertion of a functional protein has a conductance of approx 35 pA. The sequential insertion of the protein is observed as a stair-like voltage clamp trace.

### Materials and Chemicals

8×8 array on ETFE LZ200 partition

25 mg/ml DPhPC+NBD-PC (1-oleoyl-2-[6-[(7-nitro-2-1, 3-benzoxadiazol-4-yl) amino]hexanoyl]-sn-glycero-3-phosphocholine) (Avanti Polar Lipids Inc. (Alabaster, Ala., USA)

0.5 mg/ml α-hemolysin (αHL) solution diluted 20× (Sigma-Aldrich Denmark, Brandby, Denmark).

KCl buffer solution 1M

### Equipment & Required Laboratory Working Time

Inverted fluorescence microscope

BLM amplifier and signal generator: The experimental setup consisted of a Model 2400 Patch Clamp Amplifier with a head stage containing 10 G/10 M feedback resistors (A-M Systems, Inc., WA, USA) and a Thurlby Thandar Instruments model TG2000 20 MHz DDS function generator (RS Components Ltd, Northants, UK). The electrodes were placed in the trans and cis compartments of the bilayer formation chamber with the ground electrode positioned in the trans compartment. Data acquisition was done with a combined oscilloscope/analog-digital converter (ADC-212/50, Pico Technology, Cambridgeshire, UK) connected to a laptop computer.

Chamber shown in FIG. 26.

DPhPC has to be prepared the day before and stored at −20° C.

### Preparing Black Lipid Membrane Arrays:

Mount the chamber as described in example 10 and fill the centre chamber with 3 ml 1 M KCl and the outer chamber accordingly.

With the tip of a small Finnpipette, and the amount of lipid that is left when emptying the tip, “draw” the membranes on the array An air/lipid bubble is blown out from the pipette tip and positioned close to the array. The pipette tip is then swept across the array in a painting-like motion to establish BLMs across the apertures.

Repair any broken membranes with the tip while waiting for the membrane to stabilize.

### Preparation of αHL Solutions:

αHL is supplied as a lyophilized powder with a content of 0.5 mg per vial.

To make a stock solution, add 1 ml Milli-Q water to the vial with αHL, this gives a stock concentration of 0.5 mg/ml.

Dilute the stock solution with water or an appropriate buffer by 20 fold to make a 25 μg/ml working solution.

Store at −20° C. until use.

### Data Acquisition and Incorporation of αHL:

When a stable BLM array has been established (check by capacitance and conductance measurements), apply a 60 mV DC offset using the menu on the signal generator. It is convenient to store this as a program.

In pico scope set the axes for the membrane output to 20V on the γ-axis amplitude and 50 div/s on the x-axis. This gives a voltage clamp trace running for 500 s.

Leave the DC offset on for approx 5-10 min before adding the protein solution to ensure a stable membrane array. If single apertures rupture, then repair and wait for another 5-10 min with the DC offset turned on.

For protein incorporation apply 10 μl of a 25 μg/ml αHL solution to the top chamber and away from the membrane array. This gives a final αHL concentration in the top chamber of approx 83.3 ng/ml.

Follow the membrane trace on pico scope to see when protein is reconstituted into the membrane array.

Store the trace after successful protein incorporation.

### Results and Conclusion

FIG. 25 shows that αHL was successfully inserted in a composite biomimetic membrane of the invention. A) shows the bilayer array used in the αHL experiment using a 2.5× objective. B) shows a transmitted light image of a part of the bilayer array to demonstrate the prescense of bilayers, and C) shows the corresponding fluorescence image of the fluorescent NBD-PC lipid analog that is present in the bilayer forming solution. D) shows the functional incorporation of αHL proteins in the preformed bilayer array. The functional reconstitution of αHL proteins in the lipid bilayer array is observed as a stepwise increase in the conductance, where each functional incorporation results in an approx. 35 pA conductance increase. The resulting stair-like voltage diagram in FIG. 25D shows that it is possible to insert functional transmembrane proteins in lipid bilayers established across the ETFE scaffold. Moreover, the fact that single channel events can be resolved with very low background noise shows that the scaffold is applicable to sensitive membrane protein-based biosensor applications such a drug screening.

An example of the usefulness of having access to an array of functional αHL transmembrane proteins is in testing of compound libraries for modulation, such as inactivation or antagonizing, of the protein. Beta-cyclodextrin can be used as a positive control since this molecule is a known αHL antagonist (Li-Qun Gu and Hagan Bayley, Interaction of the Noncovalent Molecular Adapter, b-Cyclodextrin, with the Staphylococcal a-Hemolysin Pore. Biophysical Journal Vol. 79 October 2000 1967-1975).

### Example 13

### Preparation of Composite BPM Membrane Using the APM Method

### Materials and Chemicals

KCl, 200 mM
30 mg of 4 PMOXA(y)-PDMS(x)-PMOXA(y) (poly(2-methyloxazoline)-block-poly(dimethylsiloxane)-block-poly(2-methyloxazoline) having 4 cross-linkers. Herein is used 5=y=15; 40=x=80, which can be custom synthesized by Polymer Source, Quebec, Canada. Nardin et al. Langmuir Vol. 16, p 7708-12, 2000
Photoinitiator: DAROCUR® 1173
Solvent:
   Decane
   Choroform
   1,2 butandiol diacrylate

Equipment & Required Laboratory Working Time
APM chamber fully assembled including 8×8 scaffold Tefzel LZ200
Labware in general
90 min+membrane lifetime
Instruments for voltage-clamp measurements:
PicoScope data acquisition unit
Generator and amplifier (Eastern Scientific)

Standard Operating Procedure:

Preparation of Polymer Solution:

Weight 30 mg of polymer in a small glass vial. Place the polymer stock back to the fridge. Dissolve completely (takes some time) the polymer by adding and shaking 50 μl of Chloroform. Add 250 (between 200 and 300) μl of Decane and shake again. Using a “plastic tip” pipette, add 50 μl of 1,2 butandiol diacrylate.

Preparation of the Electrolyte Solution:

Prepare a non-filtered 50 ml solution of KCl 200 mM. Using a “plastic tip”pipette add 50 μl of photoinitiator (Darocur). With the same pipette, add one droplet of photoinitiator in the polymer solution. Shake and than filter the electrolyte solution using a 200 μm filter connected to the syringe. Wrap the polymer solution+electrolyte solution's container with aluminium foil. Mount the chamber using quartz glass slides. Use a viton ring with a large hole in front of the ETFE partition.

Rinsing the Assembled Chamber with Buffer:

Fill up the back chamber with the electrolyte solution, including the shaft space. Discard the buffer and refill up to the level just below the shaft space. Fill up, gently, the front chamber and make sure the area in front of the partition and behind the cut inner glass cover; the ‘capillary volume,’ is wetted. Discard and refill with buffer until desired level is reached.

Injecting Polymer Solution on Top of Buffer:

Inject 150 μL of polymer solution (organic phase) by inserting a Hamilton-syringe into the injection shaft and gently place the organic phase volume on top of the buffer surface. Rinse immediately the Hamilton-syringe with chloroform: otherwise the metal piston will be glued to the glass syringe. Fill up the front chamber with buffer until the surface of the ‘capillary volume’. Put the APM chamber inside a Faraday-cage.

Set Up for Doing Voltage-Clamp Measurements:

In short: Make sure the generator is on and the PicoScope data acquisition software is running. Position the head-stage mounted AgCl-electrodes in the front and back chamber. For ease, adjust the external off-set to align the blue (membrane signal) line just below the red line showing the external signal. Collect reference data for both triangular (capacitative signal) and square (resistance or conductive signal.) applied external signal.

Lowering and raising the buffer level in front of partition to form BPM's:

Use the principle of painting polymer membranes on the apertures grid. The organic phase containing polymers are painted over the partition thereby depositing and partitioning polymers into the apertures. As the aqueous phase subsequently surrounds the apertures and organic phase, the polymer molecules spontaneously self assemble into planar polymer thin membranes. Apply external signals, squared and triangular to record snapshots of the resulting electrical membrane characteristics.

Observe the membranes development for a few minutes (10-15). If the electrical signal are really low (cap<25 mV), reform the membrane by lowering and raising the buffer level. If the electrical signals evolve very fast (+/−5 mV per minute), the membrane is not stable. So it is better to reform a new one. When the electrical signals are nice and evolve slowly, cross-link the membrane following the instructions mention below.

Formation of Second ‘Generation’ BPM's:

Break first ‘generation’ membranes by switching generator power on and off. Lower the ‘capillary vol.’ and add 50 μL of the polymer solution. Raise, lower and reraise the ‘capillary vol.’ Observe that an electrical seal has been achieved which is an indicator for successful membrane formation. If the membranes break within the first 30 minutes, repeat the lowering/raising and note accordingly in the experiment journal. Perform data recording in the following series:
First 30 min. every 2 minutes
Next 60 min. every 10 minutes
Remaining time every 20 minutes

After formation of BPMs in the scaffold protein can be incorporated in the membranes according to the procedures disclosed in Ho et al. Nanotechnology Vol. 15 (2004) 1084-94 for incorporation of bacteriorhodopsin or COX in copolymer membranes, or according to Ho et al. Nanomedicine Vol 2 (2006) 103-12 for membrane insertion of OmpF solubilized in n-octylpolyoxyethylene.

Following protein incorporation and allowing for subsequent equilibration time of up to 1 hour, the membrane can be cross-linked according to the procedure below.

Polymer Cross-Linking

Since every components are already mixed, we just need to irradiate the whole chamber with UV to activate the photoinitator. Hold the UV lamp (EA-140/FE from Spectroline, 625 μW/cm² at 6 inch distance) far as possible from the chamber. Switch on the UV lamp and place it in front of the chamber. Wait 7 minutes, place the lamp as far as possible and switch it off. After 4 days the 8×8 composite membranes attained a steady state conductance value of 500 nS, and a steady state capacitance value of about 3000 pF. The composite membranes were stable for at least 6 days. Thus, we have shown that a composite biomimetic membrane where the amphiphilic membrane forming compound is a copolymer can be formed using the scaffold of the invention.

Further aspects of the invention relates to the following statements:

A membrane scaffold comprising a planar material having a hydrophobic surface (water contact angle greater than about 100°, such as a Teflon, e.g an ETFE film) wherein a central functional area comprising a plurality of apertures have been formed using an optically guided thermal process, and wherein the apertures in said film are essentially of a circular shape and have an essentially perpendicular position relative to the plane of said planar material, and further characterized in that the aperture rims are smooth and formed into bulges; and said membrane scaffold wherein the perforated area covers from about 30% to about 60% of said central functional area; said membrane scaffold wherein said apertures have a diameter of >200 μm to about 3000 μm, preferably >250 μm to about 450 μm; said membrane scaffold wherein the aperture rim further has a toroidal bulging; said membrane scaffold wherein said bulging is from about 8 μm to about 20 μm above the scaffold surface; said membrane scaffold wherein the spacing between the apertures is from about 150 μm to about 500 μm; said membrane scaffold wherein the central perforated area is about 2 cm×2 cm; said membrane scaffold where said planar material has a thickness of from about 25 μm to about 200 μm; said membrane scaffold wherein said planar material is an ETFE film having a thickness of between about 50 μm to about 75 μm; said membrane scaffold where said optical or thermal process is a CO₂ laser ablation.

A composite biomimetic membrane comprising

a) the membrane scaffold as defined in any one of the preceding claims, and

b) a biomimetic membrane formed in said apertures, where functional channel forming molecules have been incorporated in said membrane;

said biomimetic membrane wherein said channel forming molecules are selected from the group consisting of ion channel molecules, such as valinomycin and gramicidin monomers and dimers, transmembrane proteins, such as porins, aquaporin water channels, and the CD family of receptors; said biomimetic membrane wherein said channel forming molecules cover at least 1 to 10% of the bilayer area; said biomimetic membrane wherein said channel forming molecule is an aquaporin molecule, and said biomimetic membrane being useful in a filtration device for purification of a water source or a liquid, aqueous medium.

A biomimetic membrane according to any of the above statements, which is a bilayer lipid membrane wherein said lipid is selected from DPhPC and DPPC and derivatives thereof; said biomimetic membrane wherein said lipid is dissolved at a concentration of from about 10 mg/mL to about 100 mg/mL in an apolar solvent selected from hexane, octane, decane, hexadecane, etc.; said biomimetic membrane wherein said lipid bilayer further comprises a bilayer stabilising amount of cholesterol, dextran, etc.).

A filtration device for filtering essentially pure water comprising a composite biomimetic membrane according to any of the statements above.

A method of preparing a composite biomimetic membrane comprising the following steps where the reference numbers refer to FIG. 12 herein:

a) providing a two-cell chamber wherein each cell has an upper opening to allow access to the cell, and a scaffold with a central area having multiple apertures (4) according to claim 1 which provides a partition between the two cells to form a cis chamber and a trans chamber,

b) providing a partial separation (7) in the cis chamber which extends from the top of said chamber to below said central area thus forming a relatively narrow space with said scaffold (4) where a spacer (5) between said partial separation (7) and said scaffold (4) has an upper opening to allow insertion of a syringe ,

c) providing a porous support (3) which is a functional water barrier at atmospheric pressure opposite the partial separation (7),

d) providing a first volume of aqueous buffer solution in the trans chamber opposite the partial separation (7) where said volume extends above said central area of said scaffold (4),

e) providing a second volume of aqueous buffer solution in the cell having the partial separation (7) where said volume does not reach the lower level of said central area of said scaffold (4),

f) providing a volume of membrane forming solution in the space between the partial separation (7) and the scaffold (4), and

g) adding an extra volume of said aqueous buffer into said cis chamber to raise the buffer level above said central area thereby raising the membrane forming solution completely past said apertures to form a fluid membrane therein; said method may further require that elastic seals (2) and (6) are inserted between parts (1) and (3), (4) and (5), (5) and (7), (7) and (8), (8) and (9), and between (9) and the annular sealing screw, said elastic seals being made from a chemically resistant material, such as a fluoroelastomer, e.g. Viton®; said method wherein said scaffold has been pre-painted with a solution of amphiphilic lipid in a hydrocarbon solvent; said method wherein said lipid is DPhPC and where said solvent is n-decane.

The APM-1 chamber as defined herein or as shown in FIGS. 12, 12a, 13.

While the present invention has been described with reference to specific embodiments thereof, it will be appreciated that numerous variations, modifications, and embodiments are possible, and accordingly, all such variations, modifications, and embodiments are to be construed as being within the spirit and scope of the present invention. All references cited herein are incorporated in their entirety by reference. Additional aspects, features and embodiments of the invention will be more fully apparent from the ensuing disclosure and appended claims.

1.-23. (canceled)

24. A method for producing a membrane scaffold comprising the steps of:
(a) providing a planar material comprising a foil of polyethylenetetrafluoroethylene (ETFE) or a derivative thereof having a hydrophobic surface;
(b) subjecting a spot of a functional area of the planar material to a laser beam provided by a CO₂ laser having a wavelength absorbed by said planar ETFE material, and wherein said laser beam is operated at an intensity of about 3W or below, at a spot lase duration of between 1 and 7 ms, and at an off vector delay of 1000 μs;
(c) allowing the melted material to solidify around the spot, thereby forming a bulging aperture rim;
(d) displacing the planar material or the laser beam to another spot of the functional area; and
(e) repeating steps (b) to (d) until a plurality of apertures have been formed.

25. The method of claim 24, wherein a neighboring spot is subjected to a laser beam before solidification of the melted material of a previous spot and/or wherein the apertures initially produced are receiving a higher spot lase duration and/or a higher power or intensity than the subsequently produced apertures.

26. A composite biomimetic membrane comprising a membrane scaffold produced by the method of claim 24, and a biomimetic membrane provided in said apertures.

27. The composite biomimetic membrane of claim 26, wherein functional transmembrane proteins or channel forming molecules have been incorporated in said biomimetic membrane.

28. The composite biomimetic membrane of claim 27, wherein said channel forming molecules are ion channel molecules or a member of the CD family of receptors.

29. The composite biomimetic membrane of claim 28, wherein said ion channel molecules are valinomycin or gramicidin monomers and dimers.

30. The composite biomimetic membrane of claim 27, wherein said transmembrane proteins are porins.

31. The composite biomimetic membrane of claim 30, wherein said porins are aquaporin water channels, alpha-hemolysin, OmpG, phosphoporin PhoE, or a connexin.

32. The composite biomimetic membrane of claim 31, wherein the connexin is selected from the group of Cx26, Cx30, Cx32, Cx36, Cx40, and Cx43.

33. The composite biomimetic membrane of claim 27, wherein said transmembrane proteins or channel forming proteins are selected from the group consisting of: light absorption-driven transporters, ABC (ATP-binding cassette) transporters, ABC subclass A, multidrug resistance pumps, lead and mercury ion pumps, cation diffusion facilitator (CDF) protein family members, receptors, and the channel protein POR1.

34. The composite biomimemtic membrane of 33, wherein said light absorption-driven transporter is bacteriorhodopsin, rhodopsin, opsin, or a light harvesting complex from bacteria.

35. The composite biomimemtic membrane of claim 33, wherein said lead and mercury ion pump is CadA, ZntA, or MerC.

36. The composite biomimemtic membrane of claim 33, wherein said receptor is selected from the group consisting of a neurotransmitter receptor, CD-95, a receptor for serum Fas ligand, a transmembrane CC chemokine receptor, a CXC chemokine receptor, an interleukin receptor, an olfactory receptor, and a receptor tyrosine kinase.

37. The composite biomimemtic membrane of claim 36, wherein said neurotransmitter receptor is a GABA transporter, a monoamine transporter, or a glutamate transporter.

38. The composite biomimemtic membrane of claim 36, wherein the receptor tyrosine kinase is the receptor tyrosine kinase Tie-2.

39. The composite biomimetic membrane of claim 26, wherein the membrane comprises a triblock copolymer.

40. The composite biomimetic membrane of claim 26, wherein the biomimetic membrane is a lipid bilayer membrane.

41. The composite biomimetic membrane of claim 40, wherein the lipid of the lipid bilayer membrane is selected from DPhPC, DPPC, and derivatives thereof.

42. An apparatus for testing the function of a transmembrane protein or channel forming molecule incorporated in a composite biomimetic membrane of claim 27 and having the following features:
a two-cell chamber wherein each cell has an upper opening to allow access to the cell, and a composite biomimetic membrane comprising a membrane scaffold and a biomimetic membrane, which provides a partition between the two cells to form a cis chamber and a trans chamber, a partial separation (7) in the cis chamber which extends from the top of said chamber to below a functional area thus forming a relatively narrow space with said scaffold (4), a porous support layer (3) which is a functional water barrier at atmospheric pressure opposite the partial separation (7), a first volume of aqueous buffer solution in the trans chamber opposite the partial separation (7) where said volume extends above a central area of said scaffold (4), a second volume of aqueous buffer solution in the cell having the partial separation (7) where said volume does not reach the lower level of said functional area of said scaffold (4), a spacer (5) is provided between said partial separation (7) and said scaffold (4), said spacer having an upper opening to allow insertion of a syringe.

43. The apparatus of claim 42, further comprising spacers (1) and (8), glass coverslip and (9), elastic seals (2) and (6), and an annular sealing screw, wherein the elastic seals (2, 6) are inserted between the spacer (1) and the porous support layer (3), the scaffold (4) and the spacer (5), the spacer (5) and the partial separation (7), the partial separation (7) and the spacer (8), the spacer (8) and the glass coverslip (9), and between the glass coverslip (9) and the annular sealing screw, and the elastic seals (2) and (6) are composed of a chemically resistant material.

44. The apparatus of claim 43, wherein the chemically resistant material is a fluoroelastomer.

45. The apparatus of claim 44, wherein the fluoroelastomer is a fluorodipolymer.

46. The apparatus of claim 42, wherein an electrode is inserted in each of said upper openings and in contact with said first and second volumes of aqueous buffer solution.

47. The apparatus of claim 42, wherein said transmembrane molecule is alpha-hemolysin.

48. A method of testing of a compound having binding effect on alpha-hemolysin comprising:
(a) adding a solution of said compound in the apparatus of claim 42, wherein said solution is added to said cis chamber; and
(b) measuring conductance through said electrodes.
